(19) United States
(12) Patent Application Publication
(10) Pub. No.: US 2005/0250838 A1
(43) Pub. Date: Nov. 10, 2005

(54) Formulation for sustained delivery

(76) Inventors: Prasad V.N. Challapalli, Lexington, KY (US); Peddanna Gumudavelli, Lexington, KY (US); Ram B. Murty, Lexington, KY (US)

Correspondence Address: Dr. Prasad V.N. Challapalli;Murty Pharmaceuticals, Inc., 518 Codell Drive, Lexington, KY, 40509, US

(21) Appl. No.: 11/122,189

(22) Filed: May 4, 2005

Related U.S. Application Data
(60) Provisional application No. 60/568,376, filed on May 4, 2004.

(51) Int. Cl. A61K 31/405; A61K 31/165; A61K 31/343; A61K 31/137

(52) U.S. Cl. 514/419; 514/469; 514/649; 514/618

(57) ABSTRACT

Disclosed is an extended or controlled release dosage form of citalopram or its related forms and other newer antidepressants for oral administration to treat chronic patients suffering from depression and to minimize the side effects associated with the current drug treatment.

### RELATED APPLICATIONS

Priority is claimed on the basis of U.S. provisional application No. 60/568,376, filed May 4, 2004.

### FIELD OF THE INVENTION

The invention relates to a pharmaceutical formulation for the stabilization and sustained delivery of an active pharmaceutical ingredient, such as an antidepressant.

### BACKGROUND OF THE INVENTION

Mood and anxiety disorders, in their various forms and combinations, constitute a major source of personal suffering and impaired ability to engage in productive work and interpersonal relationships. Affective disorders, while characterized by depressed mood of varying degrees, exist in various forms. Mood disorders include for example depression, major depression, melancholic depression, atypical depression, minor depression, seasonal depression, bipolar effective disorder, dysthymia disorder, menstrual-related dysphoria, chronic fatigue syndrome, depression associated with somatoform disorder, fibromyalgia and treatment resistant depression. Commonly seen anxiety disorders include post-traumatic stress disorder, generalized anxiety disorder, panic disorder with and without agoraphobia, social phobia, tics, Tourette's syndrome and obsessive-compulsive disorder.

Antidepressants, including selective serotonin reuptake inhibitors (SSRI's such as citalopram, escitalopram, paroxetine, sertraline and fluoxetine) and selective serotonin and norepinephrine reuptake inhibitors (SSNRI's such as venlafaxine and duloxetine) and other newer antidepressants such as bupropion and fluvoxamine have become first choice therapeutics in the therapy of depression as well as certain forms of anxiety and social phobias. Their effectiveness in therapy may range from 50% to 60% in patients diagnosed with major depression. Antidepressants often take about two weeks to produce improvement, but may take as long as six weeks to achieve substantial benefit, and even longer for maximal benefit. Both SSRI's and SSNRI's have largely replaced tricyclic antidepressants and monoamine oxidase inhibitors (MAOIS) as first line drugs due to their more tolerable adverse effects and relative safety in over-dose. SSRI's or SSNRI's are similar to each other in effectiveness despite differences in their approval for various disorders, but patients who fail to respond to one may respond to another, possibly because of differences in tolerability. (K. Kroenke et al., JAMA, 2001, 286, 2947 and M. Fava et al., J. Clin. Psychopharmacolo. 2002, 22, 137; Treatment guidelines from The Medical Letters, Vol. 1 (11), 2003, 69; The Medical Letter, 2004, 46(1193), 81).

However, there can be problems associated with any anti-depressant treatment. Current antidepressant therapy can exhibit a delayed onset and modest proportion in achieving response or remission. For example, the response at 6 weeks to the selective serotonin reuptake inhibitor (SSRI) fluoxetine is about 50%. Remission rates with SSRI's at 8 weeks are about 35%. Delayed, incomplete and lack of response of a major depressive disorder to antidepressant therapy can be problematic for numerous reasons, including premature treatment discontinuation. Sometimes symptoms even worsen during the first weeks of therapy. In other cases, non-compliance can be related to side effects, including sexual dysfunction. Some studies suggest that non-compliance rates for antidepressants are between 30% and 60% of the patients being treated. (See Markowitz, et al. AIDS, 8, 03-412 (1994) and J. Affective Disorders, 43, 27-39, 1997).

The most common adverse effects associated with the SSRI's are nausea, diarrhea, headache, nervousness, insomnia, fatigue and sexual dysfunction; whereas with the SSNRI's are nausea, somnolence, dizziness, constipation, dry mouth, decreased appetite hyperhidrosis and some sexual side effects. Weight gain can occur with prolonged use and SSRI's could cause depressed children or adolescents to commit suicide. These may also cause mania when used to treat depression in patients with bipolar disorder (The Medical Letter, 2003, 45 (1170), 93). An increased risk of gastrointestinal bleeding, thought to be due to inhibition of platelet function has been reported (S. O. Dalton et al., Arch Intern Med 2003, 163, 59). The withdrawal effects when SSRI's are discontinued include dizziness, nausea, paresthesias, tremor, anxiety and dysphoria. They can be minimized by gradual tapering and are least likely to occur with fluoxetine followed by citalopram due to their long half life(s). Higher doses of SSNRI's can cause dose dependent increase in diastolic blood pressure. In one retrospective review, overdose appeared to be more dangerous with venlafaxine than with SSRI's (N. A Buckley and PR McManus, British Medical Journal, 2002; 325, 1332).

Since all SSRIs and SSNRI's in adequate doses are about equally efficacious, the choice among them comes down to adverse effects, drug interactions and cost. Reportedly, when initiating antidepressant therapy, nearly one thirds of patients abruptly discontinue medication. In fact this number can be as high 44% of patients who drop therapy by the third month. This adherence factor is due to the unpleasant transient side effects experienced (e.g. gastrointestinal) with them (C. B. Nemeroff, J. Clin. Psychiatry, 2003, 64(18), 25).

In particularly serious cases, non-compliance can lead to intensification of the medical condition and even potential injury to the patient or to others. Since, many of the reported side effects such as nausea and vomiting and the other discouraging parameters in continuation of the treatment are dose proportional, thus it would be highly desirable to have a dosage form of an effective antidepressant medication lasts for a longer period of time with lesser side effects.

For many therapeutics which are administered orally, it is preferred that drug molecules be released into the body at a constant, or otherwise controlled rate, over a relatively long period of time, such as, for example, 4-8 hrs or longer. The primary objective of a controlled release system has been to enhance safety and extend the duration of action. Today, controlled release systems are designed in order to produce more reliable absorption and to improve bioavailability and efficiency of delivery. Controlled release formulations have therefore been developed and marketed to improve initial therapy.

Currently, few of the SSRI's like fluoxetine (Prozac® weekly) and paroxetine (Paxil® CR) and SSNRI's like duloxetine (Cymbalta®) and Venlafaxine (Effexor® XR) are available either in delayed release pellet form or in extended release form to improve therapy for chronic patients of depression. Paroxetine CR formulations represented no change in daily dosing but it improved patient adherence with decrease in dropout rates (C. B. Nemeroff, J. Clin. Psychiatry, 2003, 64(18), 25) and fluoxetine offers convenience being once weekly to patients but still they can poses challenge in treatment due to their capacity to interact with many drugs. When interactions occur, the long half-life of fluoxetine can especially pose problems. Paroxetine may cause more weight gain, discontinuation symptoms and sexual dysfunction than the others SSRI's. Venlafaxine XR also showed no change in improving the symptoms of nausea (C. Lindsay Devane, J. Clin. Psychiatry, 2003, 64, 14) but reportedly such effects came down after the first week of treatment (Cunnigham, L. A., Ann. Clin. Psychiatry, 1997, 9, 157-164). Since, citalopram or escitalopram reportedly causes the fewest drug interactions (The Medical Letter, 2003, 45(1170), 93), with the proposed controlled form of release it can minimize the discontinuation symptoms and other side effects associated with the drug, which are dose dependent. Thus, the proposed form of delivery can be an ideal first choice in minimizing nausea, vomiting and possibly can control suicidal tendencies, a growing concern with antidepressants. It can also be a useful alternative to chronic treatment of patients or to untreated or inadequately treated patients and those patients who experience intolerable side effects with currently available dosage forms despite their responding to the therapy.

Currently, the usefulness of SSRI's alone or in combination of other drugs for treating various other disease conditions also described. This includes treatment for cardiac disorders like myocardial infarction (U.S. Pat. No. 6,372,763); and for premature ejaculation (J. Clin. Psychiatry 54, 209-212, (1993); J. Clin. Psychopharmacol. 3, 76-79, (1983); J. Clin. Psychiatry Mon. 10, 4-10, (1992); Depression 2, 233-240, (1994/1995). SSRI's have also been investigated for treatment of depression in combination with modafinil (U.S. Patent Application No. 20040229940), in combination with irindalone (U.S. Patent Application No. 20020103249), in combination with hydroxyzine (U.S. Pat. No. 5,747,494) and in combination with antipsychotic agents (U.S. Pat. No. 6,572,890; EP 0830864 A1). Additional applications include reducing alcohol consumption and evaluation for long-term treatment of alcoholism, which is currently under review (C. A. Naranjo et al., Clin. Pharm. Ther., 35, 374, 1984, and Clin. Pharm. Ther., 41, 266, 1987; C. L. Hubbell et al., Alcohol, 8, 355, 1991; C. A. Naranjo et al., J. Clin. Psychiatry, 47, 16, 1986; U.S. Pat. No. 6,071,918). Finally, SSRI's are being evaluated in combination with phentermine for the treatment of exogenous obesity (U.S. Pat. No. 5,795,895).

There are also other reports available for both SSRI's and SSNRI's describing their delivery in various dosage forms where different manufacturing procedures are adopted to make some of these active drugs as more stable or their solubility characteristics modified to meet the needs of the treatment.

To begin with paroxetine, the currently available paroxetine hydrochloride hemihydrate is only slightly soluble in water (6 to 12 mg/ml) where as an amorphous form of drug has been reported to be soluble up to 75 mg/ml at room temperature (U.S. Pat. No. 6,660,298). U.S. Pat. No. 4,721,723 (Barnes et. al.), in distinguishing the hemihydrate salt form of paroxetine disclosed therein from other forms of paroxetine, reports that such hemihydrate form is desirable because amorphous paroxetine hydrochloride is undesirably hygroscopic and has poor handling qualities.

Most of the known salts of paroxetine are considered to have unsuitable physico-chemical properties for ensuring safe and efficient handling during production of the final product, since they are unstable and possess undesirable hygroscopicity (U.S. Pat. No. 5,874,447). Furthermore, their formation by crystallization from both aqueous and non-aqueous solvents provides a low yield and contains undefined amounts of bound solvent, which is difficult to remove. A number of patents and publications have addressed the preparation of paroxetine (U.S. Pat. No. 4,007,196; Psychopharmacology 57, 151-153 (1978); Psychopharmacology, 68, 229-233 (1980); Lund, J., Acta. Phamacol. et Toxicol. 44,289-295 (1979); Eur. J. Pharmacol., 47, 351-358 (1978); U.S. Pat. Nos. 4,721,723 and 5,681,962). Further, additional publications describe the various salts forms including the anhydrous solvate form of hydrochloride salt (U.S. Pat. Nos. 5,856,493, 5,872,132 and 5,900,423), the sulfonate form also known as paroxetine mesylate having high water solubility and good thermal stability (U.S. Pat. No. 5,874,447), the maleate stable salt form (U.S. Pat. No. 6,440,459) and finally as an amorphous paroxetine hydrochloride ethanol composition which is purported to be substantially non-hygroscopic and free-flowing. The said composition is prepared by dissolving paroxetine free base in a hydrochloric acid-ethanol solution followed by drying (U.S. Pat. Nos. 5,672,612 and 6,638,948). The amorphous solid was subjected to 2.5 days drying in order to obtain a free flowing product and with some static charge, Which ultimately caused handling problems and which was said to be improved later on with solid dispersion method (U.S. Pat. No. 6,638,948).

In addition, it has been reported that tablets of paroxetine often develop a pink hue due to the limited photostability of crystalline hydrochloride hemihydrate during an aqueous granulation process and can be overcome by using a formulation process in which water is absent, such as by direct compression or by dry granulation (WO 95/16448; U.S. Pat. No. 6,113,944). The PCT publication does not mention what the coloring compound(s) are or their route of formation. Subsequent documents however reveal that the coloration problem involves the formation of a coloring dimer impurity and such formation was identified to occur if the prevailing pH condition of formulation is above 6.5 (U.S. Pat. No. 6,645,523).

The various strategies reported so far to address the above problem includes adsorbing of paroxetine or its solution onto a suitable adsorbent material such as calcium phosphate and evaporating the solvent (U.S. Pat. No. 6,699,882). Another approach is a wet granulation method where a drug solution in water or ethanol-water mixture heated to 60° C. with combination of a water dispersible polymer such as polyvinyl pyrrolidone and an acidulant such as citric acid was prepared and which was adsorbed onto a filler such as dibasic calcium phosphate or microcrystalline cellulose for obtaining amorphous form of paroxetine (U.S. Pat. No. 6,660,298). In addition, a solid dispersion comprising active drug in free base form was dissolved in volatile organic solvent and treated with hydrochloric acid in water to convert it into hydrochloride salt to which a water soluble polymer was added, and spray dried to obtain the active drug in the form of amorphous water soluble matrix. Alternatively, the commercially available hydrochloride salt form of drug was dissolved in a co-solvent of volatile organic solvent and water to which a water soluble polymer was added and spray dried to obtain the active drug in the form of amorphous water soluble matrix (U.S. Pat. No. 6,720,003). PCT International Application No. WO99/56751 also describes a process for preparing an amorphous form of paroxetine. The aqueous solvent process involves heating of solution to 60° C. due to the low solubility of the active drug where mixing of paroxetine base or salt with water and a polymer, and drying said mixture was reported. Finally, a free flowing roller compact or slug material of dry mix having paroxetine free base with microcrystalline cellulose and dicalcium phosphate dihydrate was reported (U.S. Pat. No. 6,113,944).

The other strategies include a process describing for preparing a solid dispersion of an anhydrate form of a paroxetine salt wherein the process use free base of paroxetine, an oil, dissolved into a nonaqueous solution such as ethanol containing previously dissolved portions of a water soluble polymeric carrier (e.g., polyethylene glycol or polyvinyl pyrrolidone). Then, the paroxetine free base in a solution is contacted with at least one equivalent of an acid (hydrogen chloride in the form of dry hydrogen chloride gas or dry hydrogen chloride dissolved into a non-aqueous solvent) to form a paroxetine salt in solution (paroxetine hydrogen chloride). The non-aqueous solvent is removed by evaporation under vacuum (U.S. Pat. Nos. 5,955,475 and 6,168,805). Alternatively, a second method is described wherein the paroxetine free base is dissolved into the solution of polymeric carrier and non-aqueous solvent to form a mixture which is heated to form a molten homogeneous melt of polymeric carrier and paroxetine free base. Thereafter, the molten homogeneous mixture is contacted with at least one equivalent of dry hydrogen chloride to form paroxetine hydrogen chloride in the molten homogeneous melt, and such a melt is then cooled to form a water soluble solid state dispersion of an anhydrate form of paroxetine hydrochloride (U.S. Pat. No. 5,955,475). Finally, compositions comprising of a paroxetine—amberlite IRP-88 complex in an oral liquid pharmaceutical composition (U.S. Pat. No. 5,811,436) and a bite dispersion tablet were reported (U.S. Pat. No. 6,475,510).

Therefore, in the present invention non-aqueous volatile solvent is acidified with hydrochloric acid or any other suitable acids which is soluble in such solvent along with optional antioxidants will be used for wet granulation of the paroxetine along with suitable diluents or binders to protect the drug molecule from further degradation and to help in improving the solubility of the drug molecule.

The reported controlled or delayed release formulations for paroxetine involves combination of a paroxetine and a polyethylene wax, which forms a homogeneous drug-additive composite by heating to a temperature below 150° C. (U.S. Pat. No. 5,807,574).

The various other controlled or delayed preparations for SSRI's or SSNRI's include an enteric coated controlled release bilayered tablet formulation containing a selective serotonin reuptake inhibitor (SSRI), which is either in a reaction complex formation with a calcium polycarbophil component (water-swellable, but water insoluble, fibrous cross-linked carboxy-functional polymer) or is in deposit core to which a elastic support-platform is applied (U.S. Pat. No. 6,548,084). Other approaches include an osmotic delivery system having an antidepressant drug in the form of an amorphous solid dispersion in the core surrounded by non-eroding and non-dissolving membrane (U.S. Pat. No. 6,706,283). Finally, an encapsulated product for antidepressant drugs was also reported (U.S. Pat. Appl. Nos. 20020044960 and 20020044962).

The various patents covering sertraline hydrochloride are described in various applications including its polymorphic forms (U.S. Pat. Nos. 5,248,699 and 5,734,083), as a controlled release matrix retained in the stomach (U.S. Pat. No. 6,340,475), the controlled onset and sustained release matrix core with multiple coatings (U.S. Pat. No. 6,500,459). In addition, a solid dispersion technique is described where active drug in free base form is dissolved in volatile organic solvent and treated with hydrochloric acid in water to convert it into hydrochloride salt to which a water soluble polymer was added. Further, it was spray dried to obtain the active drug in the form of amorphous water soluble matrix (U.S. Pat. No. 6,720,003). Also, a prolonged released matrix is prepared with a mixture of cellulose polymer and polyvinylpyrollidone—polyvinyl acetate (U.S. Pat. Appl. No. 2002/0132002). Finally, additional applications include a multi-functional sustained delivery of sertraline and its various salt forms (U.S. Pat. No. 6,517,866) and as microparticulate injection for long-term delivery (U.S. Pat. No. 6,482,440).

The various patents covering the venlafaxine hydrochloride include a delayed burst release after at least three hours resulting in absorption mainly through the colon over a period of at least 24 hrs (U.S. Pat. No. 6,703,044). An extended release formulation is also described in spheroids comprised of active drug with microcrystalline cellulose and optionally, hypromellose coated with a mixture of ethyl cellulose and hypromellose (U.S. Pat. Nos. 6,274,171, 6,403,120 and 6,419,958; EP 0 797 991). Additional patents describe an encapsulated extended release dosage form (WO 99/22724) and an osmotic delivery system with two layers such as a drug layer and an osmotically driven displacement layer surrounded by a water permeable/drug impermeable membrane with an exit passage for the drug (WO 94/27589). Another zero-order sustained release solid dosage form of venlafaxine comprises a matrix core having the active agent with both intragranular and extragranular ethylcellulose, Which is also covered with hydrophobic polymer such as ethylcellulose coating for encasing the entire matrix core (U.S. Pat. Appl. No. U.S. 2003/0133982).

Reportedly, the highly soluble venlafaxine hydrochloride (570 mg/ml) causes handling problems not only due to irritation to the skin and poor compressibility/capping but also in preventing solubility related rapid release from hydrogel based matrix system for extended delivery (U.S. Pat. No. 6,274,171 and EP 0 797 991A). There are a couple of other low water soluble salt forms such as maleate (U.S. Pat. No. 6,696,496) and benzenesulfonate, which is also known as besylate ((U.S. Pat. No. 6,717,015). These salts were reported to improve physical characteristics of the drug during manufacturing. Thus, the present invention proposes a suitable granulation method or treating the active drug directly with suitable excipients mainly hydrophobic in nature to slower the solubility and also to impart better handling characteristics to the active material.

The various reports available for citalopram hydrobromide or escitalopram oxalate includes preparing a solid unit dosage form by direct compression of a mixture of citalopram base or a pharmaceutically acceptable salt in a hard gelatine capsule (U.S. Pat. Appl. No. 20030109577). Other techniques include a roller compaction process for citalopram with excipients (U.S. Pat. Appl. No. 20040058989) and a melt granulated homogeneous composition comprising of one or more hydrophilic cellulose ether polymers and a polyethylene glycol as melt binder for citalopram and escitalopram (U.S. Pat. Appl. No. 20040170695). Finally, U.S. Pat. No. 6,607,751 describes a sustained or pulsatile release delivery system for citalorpam hydrobromide incorporating microbial polysaccharide, hypromellose, crosslinked polymer like carbopol® and lipophilic polymer like gelucire® and compritol® 888 ATO polymer along with diluents, glidants and lubricants. The reported system is too complex and requires many controlling agents to achieve the target release. Therefore, it is of the objective of the present invention to develop a more reliable system, which can provide an extended or controlled release system with fewer variations in plasma levels.

The reports available for other antidepressants such as fluoxetine, duloxetine or tomoxetine includes a controlled onset and sustained release granulated matrix core with multiple coatings (U.S. Pat. No. 6,500,459). An enteric formulation of duloxetine is also described in the form of pellets coated with an enteric polymer like hypromellose acetate succinate, which is due to lack of stability in acid environment (U.S. Pat. No. 5,508,276). A semisolid hydrophobic carrier matrix having hydrophilic substance capable of creating channels is presented in a capsule for sustained delivery of antidepressant drug such as tomoxetine (U.S. Pat. No. 4,847,092). Fluoxetine delivery system through intravaginal or rectal route (U.S. Pat. Appl. No. 20030133977) and an aerosol preparation for delivery of antidepressants through inhalation route are also reported (U.S. Pat. No. 6,783,753). Thus, the present invention mainly focuses on the controlled or delayed delivery of SSRI'S and SSNRI's despite their long half-life of few of the drugs, to overcome the widely reported side effects such as nausea, vomiting etc., thereby helping patients who suffer from such side effects. The controlled or delayed delivery will increase their chances of adherence to the treatment of depression and in those situations where drug has long half-life it helps to lower the daily dose requirements. Further, the invention also addresses some of the above reported physico-chemical stability issues involved with antidepressant drugs such as paroxetine, venlafaxine and duloxetine. The above said objectives are achieved in the proposed invention by presenting the drugs in a suitable controlling release matrix prepared with pharmaceutically suitable polymers either alone or in combination of erodable or by swelling type of polymers with an optional pore forming agent or an film/enteric coat wherein the core of the tablet is either directly compressed or prepared by a suitable wet granulation techniques for their successful delivery.

### SUMMARY OF THE INVENTION

It is a primary object of the invention to provide a controlled release dosage form of citalopram or its related forms and other newer antidepressants for oral administration to treat chronic patients suffering from depression and to minimize the side effects associated with current drug treatment. The invention helps in restricting the drug dosing of the proposed newer antidepressants to once daily administration or reducing further their currently prescribed daily dosage to either once, twice or thrice weekly. This will thereby poses less risk for side effects and offers convenience to patients in dosing. In addition, the developed dosage form of citalopram or its related forms especially escitalopram can alternatively be prescribed as the first choice of treatment to chronic patients for having minimal chances to interact with other drugs and relatively better safety profile over fluoxetine or paroxetine wherein the chances for later drugs to interact with other drugs are high. The controlled release of the active dosage form was achieved either by presenting the active drug within an erodable or swellable controlling release agent or both combined in a solid dosage system with an optional pore forming agent or an film/enteric coat either to control the release from the preparation or to make it stabilized to prevent any formation of degradation products. It contains at least about 80% potency of undegraded active drug after storage for three months at about 40° C. and 75% RH.

The extended or controlled release of the active dosage form is achieved by a method comprising mixing of an active drug with suitable extended or controlled release agents and other suitable excipients to either compress directly or prepared by a suitable wet granulation techniques or by coating the drug onto suitable placebo granules or to fill them into capsules of suitable size. Optionally, a film/enteric coat covering the dosage form to achieve the desired release and the above such product is stable during its shelf life. The required granules for both compression or filling into capsules can be obtained either by physical mixing or spraying to coat the active material with suitable (binders or control release) agents to offer enough strength to granules and to coat them further to facilitate the desired release profile. Optionally, the necessary ingredients of dosage form can be granulated either by aqueous or non-aqueous solvent system or hydroalcoholic systems and dried, sieved and lubricated.

Another objective of this invention is to obtain the extended or controlled release from the dosage form, which can be achieved either by eliminating the pore forming agent or any suitable diluent, or by eliminating swellable and hardness yielding low viscosity binders.

Further, it is also objective of the invention to make a stable preparation of dosage form by maintaining suitable storage conditions or surrounding environment in the matrix, which will be necessary during its shelf life or at the time of delivery. In addition, the solubility of drug either very high or low in water is manipulated wherever necessary to attain the desired level of solubility for the drug in chosen medium or to its release from the dosage form.

In brief, the core of above said dosage form can be prepared in one of the following mannerS depending on the needed drug solubility, stability or therapy requirements. The solid dosage form can be coated or enteric coated to achieve the therapeutic objectives.
1. The core of the dosage form of a drug, which is known to be unstable or develops a color or can undergo degradation if not conveniently protected or needed a taste masking effect or whose release is to be regulated in the stomach to minimize associated side effects in drugs like paroxetine or any other antidepressant known to undergo any one such change and which needs to be addressed during manufacturing can be processed by adopting to one or more of the following steps:
a. active drug is premixed with suitable excipients such as diluents, binders, solubilizers and stabilizers.
b. the above premix is granulated with volatile solvents or mixture of hydroalcoholic solutions wherein the acidifying agents are conveniently dispersed or dissolved in them to facilitate mixing and granulation.
c. the above granules are dried, sieved and blended with suitable excipients such as release controlling polymers, glidants and lubricants. Such blended granules are either compressed or filled into capsules.
d. optionally, the placebo granules can consist diluents and binders which are pretreated with above acidifying solutions and dried followed by blending with active drug, solubilizers, stabilizers, release controlling polymers, glidants and lubricants. Such blended granules are either compressed directly or filled into capsules.
e. the core units are suitably film and/or enteric coated as necessary.
2. The core of the dosage form of a drug, which is known to be poorly compressible or highly soluble or whose release is to be regulated in the stomach to minimize associated side effects in drugs like venlafaxine or any other antidepressants known to undergo any one such change and which needs to be addressed during manufacturing can be processed by adopting to one or more of the following steps:
a. active drug can be suitably premixed with either hydrophilic or hydrophobic substances such as binders, release modifying agents and lubricants.
b. the above premix is granulated with water or volatile non-aqueous solvents or mixture of hydroalcoholic solutions wherein the added drug and excipients can conveniently be dispersed or partially be dissolved in them to facilitate the mixing and granulation.
c. the above granules are dried, sieved and blended with suitable excipients such as release controlling polymers, glidants and lubricants.
d. optionally, the active drug can be sprayed onto placebo granules or spheres as a coat wherein the spraying solution contains the active drug, binders, hydrophobic release modifying agents, lubricants and dried. Such dried granules are sieved and blended with suitable excipients such as release controlling polymers, glidants and lubricants, such granules are compressed or filled into capsules.
e. optionally, the active drug is premixed with binders, hydrophobic release modifying agents and lubricants in a suitable solvent system and spray dried in a fluidized bed system to obtain coated drug particle with better compressibility characteristics, such particles can be blended with suitable hydrophilic or hydrophobic excipients such as diluents, binders, release modifying agents, glidants and lubricants. Such blended granules are directly compressed or filled into capsules.
f. optionally, active drug is premixed with suitable hydrophilic or hydrophobic excipients such as diluents, binders, release modifying agents followed by blending with glidants and lubricants. Such blended granules are directly compressed or filled into capsules.
g. the core units are suitably film and/or enteric coated as necessary.
3. The core of the dosage form of a drug, which is known to be unstable in acidic environment or light sensitive or is known to interact with enteric polymers or whose release is to be regulated in the stomach to minimize associated side effects in drugs like fluoxetine, duloxetine or any other antidepressants known to undergo any one such change and which needs to be addressed during manufacturing can be processed by adopting to one or more of the following steps:
a. active drug can be suitably premixed with either hydrophilic or hydrophobic substances such as diluents, binders, pH dependent and independent release modifying agents.
b. the above premix is granulated with water or volatile non-aqueous solvents or mixture of hydroalcoholic solutions wherein the added drug and excipients can conveniently be dispersed or partially be dissolved in them to facilitate the mixing and granulation or the obtained mass can be conveniently extruded and spheronized without applying any external heat.
c. the above granules are dried, sieved and blended with suitable excipients such as binders, release controlling polymers, glidants and lubricants, such granules are compressed or filled into capsules.
d. the core units or granules are suitably spray coated using pH dependent or independent film forming polymers as necessary. Wherein the spraying solution contains the binders, pH dependent or independent film forming polymers, lubricants, plasticizers, coloring agents, opacifying agents and dried. Coated and dried granules are sieved and blended with suitable excipients such as glidants and lubricants. Such granules are filled into capsules or directly compressed into tablets.
e. optionally, the active drug is premixed with diluents, binders, pH dependent or hydrophobic release modifying agents and lubricants in a suitable solvent system and spray dried in a fluidized bed system to obtain coated drug particle, such particles can be blended with suitable hydrophilic or hydrophobic excipients such as diluents, binders, release modifying agents, glidants and lubricants, such granules are directly compressed or filled into capsules.
f. The core units further can also be coated with film or pH dependent release modifying polymers as necessary.
4. The core of the dosage form of a drug, which is water soluble or slightly soluble and whose release is to be regulated in the stomach to minimize associated side effects for drug like citalopram, escitalorpam or any other antidepressant belongs to above such category and such issues needs to be addressed during manufacturing can be processed by adopting to one or more of the following steps:
a. active drug can be premixed with suitable excipients such as diluents, binders, solubilizers, stabilizers, swelling and/or erodable release controlling polymers followed by blending them with suitable glidants and lubricants, such granules are either compressed directly or filled into capsules.
b. optionally, the active drug is premixed with suitable excipients such as diluents, binders, solubilizers, stabilizers, swelling and/or erodable release controlling polymers followed by granulating them with water or volatile non-aqueous solvents or mixture of hydroalcoholic solutions or aqueous solutions and the obtained mass can be conveniently extruded and spheronized without applying any external heat. Such granules are sieved and blended with optional release controlling polymers, glidants and lubricants. Blended granules are either compressed or filled into capsules.
c. optionally, the active drug can be sprayed onto placebo granules or spheres as a coat wherein the spraying solution containing the active drug, binders, swelling and/or erodable release modifying agents, lubricants and dried. Such granules are sieved and blended with suitable glidants, lubricants and optional release controlling polymers. Blended granules are either compressed or filled into capsules.
d. optionally, the core units or granules are suitably spray coated using pH dependent or independent film forming polymers as necessary. Wherein the spraying solution contains the binders, pH dependent or independent film forming polymers, lubricants, plasticizers, coloring agents, opacifying agents and dried. Such granules are sieved and blended with suitable glidants and lubricants. Blended granules are filled into capsules or compressed into tablets.
e. optionally, the active drug is premixed with binders, pH dependent or hydrophobic release modifying agents and lubricants in a suitable solvent system and spray dried in a fluidized bed system to obtain coated drug particle, such particles can be blended with suitable hydrophilic or hydrophobic excipients such as diluents, binders, release modifying agents, glidants and lubricants. Such blended granules are compressed or filled into capsules.
f. the core units are suitably film and/or enteric coated as necessary.

A further aspect of the present invention for uncoated or film coated citalopram ER dosage form includes providing a release of citalopram of not more than 40% in 1 hour, not more than 70% in 4 hours and not less than 70% in 8 hours in a dissolution test conducted 37° C. in a USP Type II apparatus with a paddle speed of 75 rpm. The invention also describes a release from enteric coated dosage form of citalopram of not more than 10% in 2 hour in acid medium and not more than 70% in 4 hr and not less than 70% in 8 hours. The above release requirements are also holds true for the remaining newer antidepressants discussed in the invention.

The present invention will be described initially in connection with its applicability to citalopram. However, it has applicability to other types of pharmaceuticals proposed in the invention like newer antidepressants such as escitalopram, paroxetine, venlafaxine, duloxetine, sertraline and fluoxetine where extended release treatment can improve the therapy for chronic patients to overcome associated side effects such as nausea and vomiting and also increases their chances of adherence to treatment of depression. The invention is particularly well suited to water-soluble drugs, but can also be used for water-insoluble drugs. Presently, the reported clinical studies for citalopram are available up to a maximum dose of 60 mg per daily. Therefore, the present invention is focused mainly on developing a controlled release system covering an acceptable dose range of 10-100 mg of citalopram per day or per week as needed.

### DETAILED DESCRIPTION OF THE INVENTION

The present invention encompasses pharmaceutically active materials and pharmaceutical compositions, incorporating those active materials, which are effective in the treatment of mood and anxiety disorders, such as depression. The materials and compositions of the present invention are preferably administered by oral route.

The present invention relates to pharmaceutically active materials comprising an antidepressant compound selected preferably from the group consisting of citalopram, escitalopram, paroxetine, venlafaxine, duloxetine, fluoxetine, sertraline and other newer antidepressants such as fluvoxamine and bupropion. The hydrochloride salts forms of paroxetine, sertraline, venlafaxine, duloxetine and fluoxetine, hydrobromide form of citalopram and oxalate form of escitalopram are preferred apart from any other pharmaceutically acceptable salts of the above mentioned drugs.

Currently, the drugs considered for the proposed invention to deliver them as an extended or controlled delivery so as to facilitate an improvement in the patient compliance to the treatment of depression, are known to posses various formulation related challenges either due to the drug nature or due to the therapeutic requirements. These are identified based on the drug(s) which includes:
a. instability or development of color or impurity/degradants formation related to pH environment of the matrix (e.g. paroxetine) or
b. to avoid bitterness (e.g. paroxetine and fluoxetine) or
c. poor compressibility (e.g. venlafaxine) or
d. its photosensitivity (e.g. duloxetine) or
e. either very high solubility (e.g. venlafaxine) or poor solubility (e.g. paroxetine) in water or
f. release is to be regulated in the stomach to minimize associated side effects (e.g., citalopram, escitalopram, paroxetine, sertraline, venlafaxine and fluoxetine) or
g. lack of drug stability in acidic environment (e.g. duloxetine) or
h. is known to interact with enteric polymers to cause release control problems in the stomach and its resultant effect on low bioavailability (e.g. duloxetine, fluoxetine, paroxetine and sertraline).

Therefore, these issues need to be carefully addressed during manufacturing not only to meet their individual stability, acceptability and handling requirements for their successful delivery but also to meet the therapeutic objectives. In order to address above discussed problems various strategies are proposed in the invention.

To control the formation of undesired colored dimer impurity known to form in an aqueous alkaline environment from the paroxetine free base, is to protect the molecule in more than one approach by maintaining the surrounding environment in the matrix preferably below pH 6.5, which can be achieved in any of the following means as known in the art, these include controlling the pH of the composition to be sufficiently acidic, limiting the amount of added water remaining in the composition, limiting the presence of oxygen during storage and reducing the level of paroxetine free base impurity in the active. In this aspect, maintaining the pH below certain range in the matrix can be achieved either by inclusion of acidifying agents to the formulation or by careful selection of formulation excipients to maintain it. The inclusion of acidifying agents into the formulation matrix should be possible in one of the following manner, these include direct addition to the formulation composition for mixing purpose or dissolving them in a suitable solvent system prior to granulation or physical treatment of the blend. It is to be noted that direct addition of such acidifying agents to the formulation matrix may not be effective if the blend is going to be directly compressed due to their presence is often restricted to smaller quantities and which may not be sufficient enough to ensure homogeneity in the blend to shield the molecule against any unwanted exposure. Therefore, above such addition is usually recommended only when their amounts are high or only when more than one such agent is added to achieve the desired protection.

To summarize, it is advisable to the dissolve them prior to their addition in a suitable solvent system such as water or non-aqueous solvents or in a mixture of hydro-alcoholic solutions. Since majority of the known acidifying agents are either inorganic or organic in nature thereby, expected to dissolve in aqueous or non-aqueous solutions or their mixtures. But, in the current situation the objective is to maintain low quantities of water or moisture level in the preparation, it is better to consider those acidifying agents soluble either in non-aqueous solvents alone or mixtures of hydro-alcoholic solutions wherein the water portion is restricted to a minimum quantity, which is enough to meet the requirements of such added agent like for the solubility or to facilitate their effective distribution during the granulation step or any other involved mixing process.

Sometimes, the careful selection of excipients to maintain the pH in the desired range in a formulation matrix may not help in overcoming the problem completely unless proper precautions are taken to choose those excipients in which the moisture and associated impurity levels such as peroxides, superoxides, hypochlorites, aldehydes, formic acid, metals such as copper and iron are maintained to a minimum level (Hartauer, K. J., et. al. Pharm. Dev. Technol., 2000, 5, 303-310). Any such exercise is very much needed and more relevant to address the oxidation of drug molecule. It is widely known that many of the release controlling polymers such as cellulose derivatives, diluents and binders to carry lot of moisture along with them and whose levels can vary from manufacturer to manufacturer also. In addition, the above reactive impurities are capable of initiating a chain of propagation reactions in the solid matrix, which are known to cause degradation of the drugs such as bupropion (in-house data) and other drugs reported in publications (Waterman, K. C., et. al., Pharm. Dev. Technol., 2002, 1-32). Hence, it is advisable to incorporate some stabilizers preferably antioxidants or chelating agents to inhibit any impurity related degradation. Use of antioxidants, which can reduce formation of peroxides will be helpful but may be less effective in eliminating of peroxides already present in a dosage form. Currently, the marketed bupropion hydrochloride (Zyban® and Wellbutrin® SR) is stabilized with an antioxidant like L-cysteine hydrochloride. Whereas, the use of chelating agents such as citric acid, edetic acid, fumaric acid and malic acid are recommended for termination of any metal induced oxidation. They can be more effective when added during granulation step than in a physical mix form.

The environment of pH can play a significant role in the stabilization of drugs to oxidation. It is in general more difficult to remove an electron from a drug when it is positively charged. For this reason, drug stability against oxidation is often greater under low pH conditions, which promote protonation of drugs if protonation is possible. In the converse, higher pH conditions, which deprotonate a drug, generally make the drug more susceptible to oxidation. With the solid dosage forms, the addition of buffers can be quite complex. In order them to be more effective it is advisable to add them during wet granulation or by coating of particles using fluid bed technology.

Further, the success of any sustained release or controlled delivery of a drug is achieved by using polymeric excipients to control the rate that an active drug is introduced to the targeted delivery site. The exact mechanism by which a polymer controls the delivery of the drug is dependent on the rate of polymer hydration and swelling which is related to its molecular weight. Therefore, any process that significantly reduces the molecular weight of the polymer is likely to affect its ability to control the drug delivery. Oxidative degradation can lead to a loss in molecular weight for several polymers commonly used in controlled release applications (Waterman, K. C., et. al., Pharm. Dev. Technol., 2002, 1-32). In addition to loss in molecular weight such degradation in polymers as mentioned can produce reactive impurities and end groups to compromise the chemical stability of drugs and also their effectiveness as release controlling agents. An example of class of controlled release polymers that can degrade to compromise the drug release rate is the polyoxyethylenes, including poly(ethylene oxides) (Polyox®), poly (ethylene glycols), and poly (oxyethylene) alkyl ethers. The polyethylene oxide is usually treated with 100-1000 ppm of butylated hydroxy toluene (BHT) to reduce such degradation by the manufacturer (Dow chemicals) This antioxidant is quite effective, however, it is volatile and can be lost during any heating steps and therefore it is advisable to include an additional antioxidants to the formulation matrix to retain the polymer behavior intact (Waterman, K. C., et. al., Pharm. Dev. Technol., 2002, 1-32).

To address, the bitterness of making any drug molecule can easily be achieved by incorporation taste masking agents such as sweeteners or by the use of popular sugar derivatives known such as lactose, fructose etc. Alternatively, it can be managed by a suitable coating process to cover the core of dosage form or the granules. Such treatment helps not only in avoiding any additional need for sweeteners which often can pose problems to certain patient populations but also acts as additional barrier to protect the formulation against the effects of moisture and light during stability of the preparation. The strategy to address the poor compressibility of the drug material can preferably be achieved by wet granulation but sometimes a direct compression process can be attempted for having certain advantages like reduced processing times and costs over the former technique. In general, the use of direct compression is limited to those situations where the drug or active ingredient has a requisite crystalline structure and physical characteristics required for formation of a pharmaceutically acceptable tablet. In those cases, where the drug lacks the required crystal morphology such as venlafaxine hydrochloride, it is known in the art to include one or more excipients such as binders, fillers and glidants to impart good flow and compression characteristics (e.g., cohesiveness) to the material as a whole to be compressed. Sometimes, prior to their direct compression they are often roller compacted or compressed under high pressure to prepare slugs which will undergo milling and/or sieving techniques to obtain ultimately the desired particle or granule size for their final compression with suitable compression aiding agents. Another limitation of direct compression as a method of tablet manufacture is the size of the tablet. If the amount of active ingredient is high, it is advisable to choose the wet granulation process for the active ingredient with other excipients to obtain an acceptable size of tablet with the desired compact strength. At the same time, the amount of filler, or binder needed in wet granulation is less than that required for direct compression. Thus, wet granulation as compared to direct compression had certain advantages like first, this method has a greater probability of overcoming any problems associated with the physical characteristics of the various ingredients in the formulation, thereby providing a material which has the requisite flow and cohesive characteristics necessary to obtain an acceptable solid dosage form. Secondly, it provides the material to be compressed with better wetting properties, particularly in the case of hydrophobic drug substances. The addition of a hydrophilic excipient makes the surface of a hydrophobic drug more hydrophilic, easing the solubility issues during testing and vice versa for highly soluble drug such as venlafaxine hydrochloride to contain the dose-dumping effects in extended release preparations. Thirdly, the content uniformity of the solid dosage forms is generally improved and thus segregation of drugs from excipients is avoided due to different physical characteristics such as density whereas the segregation could be a potential problem with direct compression. Finally, the size and shape of particles to be compressed are optimized through the wet granulation process. The issue of very high solubility with drug such as venlafaxine often can cause problems in the making of hydrogel based extended release preparations mainly due to dose-dumping effects. These dumping effects can often aggravate the situation of drug associated side effects and the problem can become much serious if the dosage form designed meant to carry more than a single dose amount to achieve extended delivery. To obtain a successful delivery system with much regulated release rates to avoid the dumping effects, can be achieved in more than one approach as known in the art. In addition to the problem of slowing the release rate the above drug in topic, has poor compressibility characteristics. Hence, it becomes obvious first choice to go for wet granulation process to meet the desired objectives. Wherein it is possible to granulate them with suitable binders and with those excipients are of hydrophobic nature to coat the drug particles sufficient enough to manipulate their wetting characteristics in presence of water. Such granulated particles can be treated or coated further with release controlling polymers to ensure better control over drug release or it can be blended with suitable excipients to facilitate their filling into capsule or to compress them into tablets. Alternatively, a spraying solution having the active ingredient, hydrophobic release controlling polymers, binders and lubricants can be prepared in a suitable solvent system and such solution can be sprayed directly in a fluidized bed system to achieve a material with better compressibility characteristics for further processing or can be sprayed onto any placebo granules such as sugar spheres or onto any extruded/spheronized particles. It is also possible to give multiple such coating layers to achieve the desired release rate during delivery and in which the subsequent layers may or may not have the drug in the spraying solution depending on the release requirements.

Similarly, it is possible to improve solubility of poorly or slightly soluble drugs by incorporation of suitable solubilizing agents such as hydrophilic polymers and complex forming agents to granulate them or such solutions can be sprayed onto suitable placebo granules or sprayed directly in a fluidized bed system to obtain free flowing amorphous drug mixtures or coated drug particles which will have enhanced solubility. But, in the present situation, the above problem of lack of high solubility to an extent is advantageous due to many of the side effects associated with these newer antidepressants are dose proportional. Therefore, any additional process adopted to improve the solubility of the drug further may result in dose dumping effects, which can actually work against addressing the core problem of the invention unless a demand for such requirement exists due to their preferential absorption form the upper gastrointestinal regions.

The final strategy to regulate the release of drug in the stomach either due to stability reasons or to minimize the side effects associated with currently prescribed newer antidepressants can addressed by giving suitable pH dependent polymer coating layers onto the core units of dosage form or can be sprayed directly in a fluidized bed system to obtain coated drug particles as known in the art. The film properties of such polymers can be manipulated by incorporation of one or more pH dependent polymers or in combination of pH independent release controlling polymers or often can be mixed with suitable pore forming agents to facilitate their release in a controlled manner in the desired regions of gastrointestinal tract to meet their preferential absorption requirements. The above such strategy can also be extended to those situations where known interactions occur between the drug and enteric polymer, to avoid such interaction a separating layers can also be included in between the core and enteric film layer and the similar separating layer can also help in forming a effective adsorptive enteric layer onto the core units.

In addition to the above mentioned challenges specific to the drug molecule considered in the proposed invention there are few more issues needs to be addressed in any formulation development especially the drug—excipient interactions so as to identify the suitable excipients necessary for their successful delivery. Reportedly, the lactose being a reducing sugar interacts with fluoxetine over time both at room temperature and under accelerated stability conditions (U.S. Pat. No. 5,910,319).

In summary, the protection of a drug molecule according to the invention includes not only minimization of potentially deleterious drug—excipient interactions, but by making drug molecule more stable where necessary by treating with various stabilizers apart from choosing an ideal packaging system with sufficient protection to drug molecule against permeation of moisture, oxygen, light and by any other agents known to degrade the molecule(s). Protection of the drug with various stabilizers involves a mechanism of shielding the drug molecule from degradation through the use of reductants, chelators, complexing agents or substances helpful in maintaining a low pH environment recommended for drug stability by various reported studies (Walters, S. M., J. Pharm. Sci., 1980, 69(10), 1206-1209).

Therefore, the present invention provides an extended or controlled release formulation for delivery of citalopram or escitalopram or any other newer antidepressants for oral administration, said formulation comprising:
l. a core consisting of active drug, controlled release agent, suitable diluent/binder, glidants and lubricants and optional stabilizer or solubilizer or pore-forming agent
a. extended release dosage form can have any combination of the following
i. swelling and erodable polymer with optional pore forming agent
ii. erodable polymer alone or in combination with optional pore forming agent
iii. erodable and swelling polymers
iv. swelling polymers mixture
b. the active drug may be presented in the dosage form
i. as premix with suitable excipients of hydrophilic or hydrophobic nature such as diluents, binders, solubilizers, stabilizers, pH dependent and independent swelling and/or erodable release modifying agents and lubricants.
ii. wherein such mixture (i) is blended with glidants, lubricants and optional release controlling agents, which is compressed directly or filled into capsules.
iii. wherein such mixture (i) is granulated with volatile solvents or mixture of hydroalcoholic solutions alone or optionally acidifying agents of organic or inorganic nature are conveniently dispersed or dissolved in the solution. Granules are dried, blended with glidants, lubricants and optional release controlling agents, which is compressed directly or filled into capsules.
iv. optionally, the granulated mass (iii) can be conveniently extruded and spheronized without applying any external heat along with pretreated granules, release modifying agents, solubilizers, stabilizers, glidants and lubricants, which are blended together and compressed directly or filled into capsules. Wherein, the pretreated placebo granules are prepared with suitable diluents and binders with acidifying solutions mentioned in (iii) and dried.
v. as spray coated onto placebo granules or inert spheres or to control the release wherein the spraying solution contains the active drug, binders, release modifying agents, lubricants and dried. Coated granules are blended with glidants, lubricants and optional release controlling agents, which are compressed directly or filled into capsules.
vi. as spray coated particles along with suitable binders, pH dependent or independent release modifying agents and lubricants, such particles can be blended with hydrophilic or hydrophobic excipients such as diluents, binders, glidants, lubricants and optional release modifying agents. Such granules are directly compressed or filled into capsules.
vii. optionally, the core units or granules mentioned in (ii-vi) are suitably spray coated using pH dependent or independent film forming polymers wherein the spraying solution contains the binders, pH dependent or independent film forming polymers, lubricants, plasticizers, coloring agents, opacifying agents and dried. Coated granules are blended with suitable glidants and lubricants, which are compressed directly or filled into capsules.
viii. optionally, the core dosage forms mentioned in (ii-vii) are suitably film and/or enteric coated to act as barrier for release or to protect against any color development/degradation products formed due to environmental factors or to mask the taste or to retard the release in acid medium.

The invention provides an extended release preparation of a newer antidepressant formulation for oral administration; said formulation can also comprise stabilizers to inhibit or prevents the degradation of active drug such as paroxetine. Stabilizers useful in accordance with the present invention maintain at least about 80% of the potency of the drug and preferably over 90% of potency after three months storage at 40° C./75% RH. The mechanism of stabilizing the active drug by chosen stabilizers involves shielding the drug molecule by their anti-oxidative property, chelating action, complex formation or they are helpful in maintaining a low pH environment around the drug molecule. The chosen stabilizers were preferably solubilized in a suitable solvent system such as hydro-alcoholic mixtures or in non-aqueous solvents and used to treat the drug particles or excipients. In some situations, such as when the stabilizer is hydrophobic in nature, treatment/coating of drug particles with stabilizer solutions can influence the solubility/release of the drug in dissolution medium. In those conditions, a solution of such stabilizer can be used initially to treat/coat the suitable excipients of the formulation and further it can be granulated/blended with other ingredients and active drug as needed. Stabilizers are often directly mixed with active drug and other excipients of the formulation to achieve a controlled release dosage form. Preferred stabilizers according to the invention are acidifying agents such as acetic acid, adipic acid, aspartic acid, citric acid, edetic acid, fumaric acid, hydrochloric acid, lactic acid, nitric acid, malic acid, maleic acid, oxalic acid phosphoric acid, phthalic acid, sulfuric acid, succinic acid, tartaric acid, toluene sulfonic acid, and other stabilizers such as alpha tocopherol, ascorbic acid, ascorbyl palmiate, benzoic acid, butylated hydroxy anisole (BHA), butylated hydroxy toluene (BHT), cysteine hydrochloride, cysteine di hydrochloride, cyclodextrins, disodium edetate, glycine hydrochloride, ion exchange resins, isoascorbic acid, propionic acid, propyl gallate, monothioglycerol and sodium metabisulfite. It is possible to use other stabilizers known in the art depending on the specific needs of the particular application and the nature of the drug. The proportion of stabilizer(s) in a formulation according to the invention is from about 0.001% to about 10% w/w, and preferably from about 0.001% to about 5% w/w.

The stabilized active drug molecule formed by mixing according to a procedure as described above, is used for making a controlled release formulation of the active drug molecule after mixing with pharmaceutically acceptable excipients. The mass of active pharmaceutical agent, whether it be in a pure form, a salt form or in a complex, ranges from 5 mg to 500 mg. The proportion of active pharmaceutical agent in a final dosage form, said final dosage form consisting essentially of active pharmaceutical agent and excpients, is from about 10% to about 80% w/w, and preferably from about 15% to about 60%. Optionally, active drug was added to pretreated placebo granules with above mentioned stabilizers and blended with necessary excipients to achieve the controlled release preparation.

A wet granulation procedure was followed according to the invention for those formulations needs manipulation of their solubility such as paroxetine or venlafaxine to meet the release requirements or in those situations where the drug has poor compressibility characteristics such as venlafaxine hydrochloride, is preferably granulated with a binder to improve the hardness of the formulation. In addition, other excipients were also included at the time of granulation such as either solubilizer to improve the solubility or hydrophobic substances to retard the rapid release. Similarly, a wet granulation procedure was also adopted for those drugs such as duloxetine, fluoxetine or other newer antidepressants wherein a pH dependent release become essential to meet the drug stability or to minimize the associated side effects with their administration. Water or hydroalcoholic or non-aqueous solutions were used as granulating fluid in the invention. Optionally, the granulated mass having with or without drug can be passed through extruder/spheronizer to obtain spheroids, which can be coated with solutions of release controlling polymers in a fluidized bed process. The active ingredient can be included in such coating solutions as an option.

A direct compression method can also be adopted in those situations wherein the drug or its treated particles have good compressibility characteristics and whose recommended dosage amounts are not very high to handle them easily without any limitations on dosage form shape and size. The various excipients such as diluents, binders, release modifying agents, solubilizers, stabilizers, glidants and lubricants can also be included during the compression step to achieve a controlled release preparation having any newer antidepressant mentioned in the invention.

A binding agent according to the invention is water soluble or water insoluble, and should possess high adhesivity and an appropriate viscosity, to guarantee good adhesion between the drug particles and other added excipients of the formulation. According to the invention, such a binding agent also functions as a diluent in that it acts to impart cohesive qualities to the material within the formulation and also to increase the bulk density of the directly compressible formulation to achieve an acceptable formulation weight for direct compression or it can act be added to mask the taste or used to modify the solubility/release as necessary or a formulation which need to be maintained below a certain pH range to maintain the product stability requirements. In situations where there is a need for either immediate availability of active drug, a pore-forming agent is also included in the dosage form. A main function of such a pore-forming agent is to create a pore in the matrix predominantly due to the freely soluble nature of such an agent in aqueous medium or in the gastrointestinal contents.

Therefore, a diluent according to the invention can posses any of the following properties such as binder, pore-forming agent, taste masking agent, stabilizer, solubilizer or release modifying agent. The following materials commonly known in the art to perform/exhibit one such function include acacia, alginic acid, acetyltributyl citrate, acetyltriethyl citrate, beeswax, chitosan, carbomer, carageenan, carnauba wax, cellulose acetate, cetostearyl alcohol, cetyl alcohol, cellulose acetate phthalate, colloidal silicon dioxide, cyclodextrins, dibasic calcium phosphate anhydrous, carboxymethylcellulose sodium, carboxymethylcellulose calcium, crospovidone, calcium carbonate, dextran, dextrins, dextrose, ethylcellulose, fructose, guar gum, gelatin, glyceryl behenate derivatives like compritol® 888 ATO, precirol® ATO 5, gelucire® 44/14, gelucire® 50/13, glyceryl mono oleate, glyceryl mono stearates, hydroxypropyl cellulose, hydroxypropyl ethylcellulose, hydroxyethyl cellulose, hydroxyethyl methylcellulose, hypromellose, hydrogenated vegatable oil, kaolin, lactose, mannitol, maltitol, maltose, medium chain triglycerides, maltodextrin, microcrystalline cellulose, methylcellulose, magnesium stearate, magnesium aluminum silicate, microcrystalline wax, povidone, polyethylene oxide, powdered cellulose, substituted copolymers of polyvinylacetate—polyvinyl pyrrolidone, poloxamer, polydextrose, polacrilin resin, polyethylene glycol, polyoxyethylene castor oil derivatives, polyvinyl alcohol, polymethacrylates, propylene glycol alginate, pregelatinized starch, starch, stearic acid, stearyl alcohol, sucrose, saccharin, silicified microcrystalline cellulose, sorbitol, sugar spheres, sodium stearyl fumarate, sodium alginate, tragacanth, trehalose, talc, xanthan gum and xylitol. The total percentage of their addition to the formulation as binder either individually or in combination is from about 1% to about 70% w/w, and preferably from about 5% to about 55% w/w. The total percentage of optional pore former either individually or in combination is from about 1% to about 30% w/w, and preferably from about 2% to about 10% w/w.

The extended or controlled release of active drug from a formulation according to the invention is achieved using individual or a combination of excipients known in the art to perform as barrier-forming polymer(s), erodable or insoluble material(s). Such excipients include hydroxypropyl cellulose, hydroxypropyl ethyl cellulose, hydroxyethyl cellulose, hydroxyethyl methy cellulose, methylcellulose, sodium carboxymethylcellulose and calcium carboxymethylcellulose, ethylcellulose, hypromellose, polyethylene oxides of molecular weight 100,000-7,000,000 Daltons (Polyox®), polyvinyl alcohols (MW: 20,000-200,000 Daltons), substituted copolymers of polyvinyl acetate and polyvinylpyrrolidone like kollidon® VA64 and kollidon® SR, sodium alginate, carrageenan, carbomer such as carbopol 71G, 971P, 934P and 974P, xanthan gum individually or in combination of ceratonia, locust bean gum or veegum, guar gum, gellan gum, chitosan, dextrates, dextrins, eudragit® (RL, RS and NE grade and pH dependent release polymers such as E, L and S grade and their mixtures), cellulose acetate, cellulose acetate trimellitate, cellulose acetate butyrate, cellulose acetate propionate, cetostearyl alcohol, cetyl alcohol, glyceryl behenate derivatives like compritol® 888 ATO, precirol® ATO 5, gelucire® 44/14, gelucire® 50/13, glyceryl mono oleate, glyceryl mono stearates, glyceryl palmito stearates, lecithin, medium chain triglycerides, eudragit® RSPO, eudragit®RLPO, stearic acid, stearyl alcohol, hydrogenated vegatable oil, carnauba wax, microcrystalline wax and beeswax. The total amount of controlled release agent present in the dosage form either alone or in combination is from about 5% to about 75% w/w, and preferably from about 5% to about 60% w/w. The possible mechanism involved in drug release is controlled by variables such as surface area and diffusion rate in a dynamic process. Barrier-forming polymers form a dynamic hydrophilic matrix system to allow for slow release of drug in a patient's body. Upon exposure to water, these barrier-forming high viscosity materials will hydrate and swell rapidly to form a hydrogel, which expands with time into the interior of the tablet allowing for diffusion of the drug from the tablet core slowly to facilitate for controlled drug delivery. Further, such effects are supported by insoluble materials of swellable or non-swellable in nature, which can acts as barrier or shielding the drug molecule under their hydrophobic coat to discourage the drug diffusion out of gelled matrix and to an extent it also helps in slowing down the matrix erosion process and thus delaying the overall dissolution rate of drug from the dosage form.

A preferred glidant or lubricant according to the invention is colloidal silicon dioxide, talc, magnesium stearate, calcium stearate, stearic acid or sodium stearyl fumarate. The proportion of glidant and lubricant present in the dosage form is from about 0.5% to about 3% w/w.

The manufacturing process for the core is either by wet granulation, dry granulation or direct compression, and where applicable involves mixing the necessary ingredients of the respective formulations according to the strategies discussed earlier, preferably in a high shear mixer granulator (GMI, India) or planetary mixer (Hobart, USA) to obtain homogeneity. If necessary, the drug is mixed with diluents prior to any granulation step. In such case, the premixed blend is then granulated with water or hydroalcoholic mixture or volatile non-aqueous solutions preferably such as ethanol, methanol, isopropyl alcohol and dried in a fluidized bed dryer (Gansons, India) or tray dryer (Kothari, India) as required. Optionally, the granulating fluid can be acidified or stabilizers dissolved in them before their addition to the granulating mixture.

Alternatively, the placebo granules are prepared in the above manner or the granulated mass with or without drug can be passed through extruder and speheriodizer (Umang, India) and dried. The dried granular mass is milled and then mixed with any other excipient (such as any diluent or controlled release agent) not added during granulation. Glidants are blended with the resulting mixture in a V-blender (Patterson-Kelley, USA) or double cone blender (Gansons, India) or octagonal blender (Gansons, India), which is capable of functioning under preferred low shear conditions followed by addition of lubricants. The lubricated mass is then compressed into tablets using a tablet press (Cadmach, India) or filled into capsule shells of suitable size using a semi-automatic capsule filling machine (Pam, India) or manual filling machine (Pam, India). The capsule shell preferably comprises gelatin, starch, chitosan or hypromellose.

A dosage form core or its granules according to the invention is preferably film-coated with a coating material such as acetyl tributyl citrate, acetyl triethyl citrate, cellulose acetate hydroxypropyl cellulose, hypromellose, a methacrylate copolymer such as eudragit® E100 or eudragit®EPO, eudragit® 30D, eudragit® (E, L and S series and their mixtures), kollicoat® IR or kollicoat® protect, kollicoat® SR 30D, EMM 30D, MAE 30 DP or MAE 100P, opadry® II, opadry® AMB and ethylcellulose (Surelease®) for identification, taste masking, aesthetic purpose, to achieve pH dependent or independent release and for stability against moisture or light. Many polymers have been investigated for use in film-coating. Most film coats are prepared by deposition of one or more film-forming polymers resulting in coats that usually represent no more than about 1-15% by weight of the final coated product depending on the need of such requirement. A coating solution according to the invention preferably contains, in addition to the film-former, a plasticizer, a glidant and an opacifying agent or a coloring agent, and a solvent system, which is composed of aqueous, hydro-alcoholic or non-aqueous solvent mixture with stabilizer(s), as described above, optionally added to such a coating solution. Some coating materials are readily available in the form of a premix with all the necessary agents required for achieving a smooth and uniform film with necessary amount of deposition to protect the core against the moisture, light or to achieve the desired release pattern. The preferred amount of each coating material is as follows: film coating material, from about 5% to about 20% w/w; plasticizer, from about 0.1% to about 2% w/w; opacifying agent, from about 0.1% to about 10% w/w; glidant/antitacking agent, from about 0.1% to about 10% w/w; optional stabilizer or pore former, from about 0.001% to about 5% w/w. Optionally, it is also possible to dissolve the active ingredient in any of those film forming solutions in combination of suitable binders, pore former, stabilizers and lubricants, which are sprayed directly in a fluid bed coating system to achieve coated drug particles or they can be sprayed onto placebo granules and dried. Such dried particles will be processed further for making of tablet or capsule as necessary.

A dosage form core or its granules according to the invention is preferably coated either with enteric polymers or pH dependent polymers to retard release preferably during the initial period of transit or to target for other regions of the gastrointestinal system. Such release retarding coat is applied either directly onto the core of the dosage form or applied over a film coat, which can not only act as sub-coat to protect the dosage form against moisture but also helps in binding of the enteric film firmly and uniformly to the dosage form. Examples of suitable enteric polymers include hypromellose phthalate, hypromellose acetate succinate, cellulose acetate phthalate, cellulose acetate trimelliate, polyvinyl acetate phthalate based dispersions like Opadry® enteric, Sureteric®, Acryl-Eze®, pH dependent release polymers like eudragit® (E, L and S series or their mixtures) either directly or after mixing with suitable plasticizers, glidants, opacifying agents or coloring agents wherein the coating solutions is prepared in aqueous, hydro-alcoholic or non-aqueous solvent mixtures with stabilizer(s) optionally added to such coating solutions. Optionally, it is also possible to dissolve the active ingredient in any of those film forming solutions in combination of suitable binders, pore former, stabilizers and lubricants, which are sprayed directly in a fluid bed coating system to achieve coated drug particles or they can be sprayed onto placebo granules and dried. Such dried particles will be processed further for making of tablet or capsule as necessary.

In coating solutions other than readily available, incorporation of suitable plasticizers into the polymer matrix effectively reduces the glass transition temperature, so that under ambient conditions the films are softer, more pliable, and often stronger, and thus better able to resist the mechanical stress, which otherwise forms a hard, non-pliable and rather brittle, properties which could be somewhat restrictive in film coating since the coated dosage form may be subjected to a certain amount of external stress. Examples of suitable plasticizers include dibutyl sebacate, diethyl phthalate, triethyl citrate, tributyl citrate, castor oil and triacetin. A preferred plasticizer, opacifying agent and glidant according to the invention are triethyl citrate, titanium dioxide and talc respectively, which are added to a coating solution prepared freshly by combining them with necessary film forming materials apart from suitable coloring agents. The preferred amount of each coating material is as follows: release retarding or enteric coating material, from about 5% to about 20% w/w; plasticizer, from about 0.1% to about 2% w/w; opacifying agent, from about 0.1% to about 10% w/w; glidant/antitacking agent, from about 0.1% to about 10% w/w; optional stabilizer, from about 0.001% to about 3% w/w.

A dosage form core according to the invention is coated in a pan coater or fluidized bed system (Ganscoater, India). An aqueous or a mixture of an organic and aqueous solvent or a mixture of organic solvent is used for film or enteric coating. Examples of suitable organic solvents are e.g., ethanol, methanol, methylene chloride, isopropyl alcohol and with or without water. The dosage form core is coated until appropriate weight gain achieved like 1-4% w/w in the case of film coating and approximately 5-15% w/w for enteric coated tablets. The tablets are dried or allowed to curing as needed at the end of each coating process. The operational parameters are maintained according to the manufacturer recommendations.

A proposed newer antidepressant formulation according to the invention, for sustained or controlled release delivery of citalopram or other newer antidepressants, is suitable for either once daily, or twice or thrice weekly administration as needed by the therapy or the patient in need. A dosage form according to the invention is, for example, a tablet or a capsule containing from about 5 mg to about 300 mg of active drug and is evaluated for performance in dissolution and assay at various intervals during stability studies. Stability studies are conducted as per the ICH guidelines. The dissolution test is conducted at 37° C. using USP Type II (Hansons Research, USA) apparatus. The once daily, twice or thrice weekly uncoated or film coated formulations of citalopram hydrobromide are tested using 900 ml of water or 0.1N HCl or phosphate buffer (0.2M, pH 6.8) as dissolution medium at 37° C. The enteric coated or pH dependent release systems are tested at 37° C. according to the procedure recommended by USP 27 for enteric-coated dosage forms.

Any of a wide variety of therapeutically active agents is formulated into a formulation according to the present invention. A therapeutically active agent according to the invention is a water soluble drug or a slightly water soluble drug. A therapeutically active agent according to the invention is, for example, antidepressants like amoxapine, maprotiline, clomipramine, dothiepin, reboxetine, amineptine, gepirone, trimipramine, nefazodone, trazodone and mirtazapine. Antipsychotic, hypnotic and sedating drugs like loxapine, mesoridazone, molindone, olanzapine, pimozide, quetiapine, risperidone, ziprasidone, mazapertine, pipamperone, selfotel, seroquel, sertindole, sulpiride, zolpidem and temazepam. Anticonvulsant and antiepileptic drugs like divalproex, gabapentin, lamotrigine, levetiracetam, primidone, tiagabine, topiramate, valpromide and vigabatrin. Antimigranine drugs like sumatriptan, frovatriptan, almotriptan, naratriptan, rizatriptan, zolmitriptan and alniditan. Angiotensin agents like benzapril, bezaprilat, enalaprilat, fosinopril, fosinoprilat, ramipril, ramiprilat, candesartan, irbesartan, telmisartan and valsartan. Antiarrhythmic agents like disopyramide, mexiletine, tocainide, flecainide, propafenone and sotalol. Betablockers like atenolol, betaxolol, bisoprolol, carteolol, nadolol, penbutolol and pindolol. Calcium channel blockers like amlodipine, felodipine, isradipine, nicardipine, nimodipine, nisoldipine and bepridril. Hypoglycemic drugs like glimepiride, glipizide, glyburide, miglitol, repaglinide, nateglinide, pioglitazone and rosiglitazone. Narcotic antagonists like alfentanil, buprenorphine, hydromorphine, morphine, methadone, oxycodone, remifentanil, pentazocine and propoxyphene. Anti-inflammatory agents like fenoprofen, oxaprozin, ketorolac, tolmetin, diflunisal, meloxicam, piroxicam, celecoxib, rofecoxib and valdecoxib.

The following materials have been used in preparing embodiments of the invention and can be replaced with similar materials available commercially and known in the art:
Hydroxypropyl cellulose (HPC, Klucel®, EXF or HXF grade, Hercules)
Polyvinylacetate-polyvinyl pyrrolidone copolymer (Kollidon® SR, VA 64 grades, BASF)
Sodium Carboxy methyl cellulose (Na. CMC, 7HXF, Hercules)
Hydroxyethyl cellulose (HEC, Natrosol® 250 HHX, Hercules)
Methylcellulose (Methocel® A4M, DOW)
Polyethylene Oxide (Polyox®, WSR 303 NF, DOW)
Ethylcellulose (ECT10, Hercules and other grades)
Polyvinyl alcohol (PVA, 30,000-70,000 MW, Sigma)
Lactose (DCL21, DMV and other grades)
Magnesium Stearate (Malinckrodt)
Colloidal Silicon dioxide (Cab-o-sil®, Cabot)
Opadry® (film coating material, Colorcon)
Sureteric® (enteric coating material, Colorcon)
Eudragit® (E, L, S, RL, RS, NE 30D grades and their mixtures)
Carbopol® 71 G (Noveon)
Precirol® ATO 5 (Gattefosse)
Cellulose acetate (Eastman)
Starch (Rpquette)

### EXAMPLES

The examples are described for the purpose of illustration and are not intended to limit the scope of the invention.

The ER dosage forms have been mainly classified as three types. They are mainly compositions which contain a combination of (i) swelling and erodable polymer with pore forming agent (Table 1), (ii) erodable polymer and pore forming agent (Table 2) and (iii) erodable and swelling polymer combination or mixture of swelling polymers (Table # 3) apart from drug and other excipients for making the dosage form.

TABLE 1
Citalopram 80 mg ER tablets

| | Example #1 | Example #2 | Example #3 |
|---|---|---|---|
| Composition | (mg/tab) | (mg/tab) | (mg/tab) |
| Citalopram hydrobromide | 100 | 100 | 100 |
| Lactose, anhydrous | 137.5 | 130 | 80 |
| HPC | 100 | 120 | 100 |
| Polyox ® | 52.5 | — | — |
| HEC | — | 40 | — |
| Na CMC | — | — | 110 |
| Magnesium stearate | 5 | 5 | 5 |
| Cab-o-sil | 5 | 5 | 5 |
| Total | 400 | 400 | 400 |

Procedure: Citalopram hydrobrmoide, lactose, HPC, ER polymer (Polyox or HEC or Na.CMC) were sifted through #60 mesh separately and mixed according to their compositions mentioned in examples 1-3 in a hobart planetary mixer for 10 min. Sifted cab-o-sil through #80 mesh was added to above blend and mixed for 5 min followed by addition sifted magnesium stearate (#80 mesh) and lubricated the granules for 2 min. The blend was directly compressed into tablets on rotary tablet press (Cadmach, India) as per the target weight into suitable shape. The uncoated tablets were tested for both assay and dissolution as per the specifications.

TABLE 2

| | Example #4 |
|---|---|
| Composition | (mg/tab) |
| Citalopram hydrobromide | 100 |
| Lactose, anhydrous | 115 |
| Kollidon ® SR | 175 |
| Magnesium stearate | 5 |
| Cab-o-sil | 5 |
| Total | 400 |

Procedure: Citalopram hydrobrmoide, lactose, Kollidon® SR were sifted through #60 mesh separately and mixed in a hobart planetary mixer for 10 min. Sifted cab-o-sil through #80 mesh was added to above blend and mixed for 5 min followed by addition sifted magnesium stearate (#80 mesh) and lubricated the granules for 2 min. The blend was directly compressed into tablets on rotary tablet press (Cadmach, India) as per the target weight into suitable shape. The uncoated tablets were tested for both assay and dissolution as per the specifications.

TABLE 3

| | Example #5 |
|---|---|
| Composition | (mg/tab) |
| Citalopram hydrobromide | 100 |
| HPC | 110 |
| ECT10 | 180 |
| Magnesium stearate | 5 |
| Cab-o-sil | 5 |
| Total | 400 |

Procedure: Citalopram hydrobrmoide, HPC, ECT10 were sifted through #60 mesh separately and mixed according to their compositions mentioned in examples 5 and 6 in a hobart planetary mixer for 10 min. Sifted cab-o-sil through #80 mesh was added to above blend and mixed for 5 min followed by addition sifted magnesium stearate (#80 mesh) and lubricated the granules for 2 min. The blend was directly compressed into tablets on a rotary tablet press (Cadmach, India) as per the target weight into suitable shape. The uncoated tablets were tested for both assay and dissolution as per the specifications.

Citalopram 20 mg, 40 mg and 60 mg Controlled release tablets. The compositions illustrated in example 1, 4 and 5 were considered based on the dissolution data (Tables 7 and 8) for making various strengths of citalopram hydrobromide extended release preparations. The required strength of each dosage form was prepared from the granules of respective composition mentioned in examples 1,4 and 5 and were compressed into tablets of different shapes with required target weights on a dose proportional basis to obtain 20 mg, 40 mg and 60 mg of active citalopram.

TABLE 4
Citalopram 60 mg ER tablets

| | Example #6 | Example #7 | Example #8 |
|---|---|---|---|
| Composition | (mg/tab) | (mg/tab) | (mg/tab) |
| Citalopram hydrobromide | 75 | 75 | 75 |
| Lactose, anhydrous | 103.125 | 86.25 | — |
| HPC | 75 | — | 97.5 |
| Polyox ® | 39.375 | — | — |
| Kollidon ® SR | — | 131.25 | — |
| ECT10 | — | — | 120 |
| Magnesium stearate | 3.75 | 3.75 | 3.75 |
| Cab-o-sil | 3.75 | 3.75 | 3.75 |
| Total | 300 | 300 | 300 |

Procedure: The previously prepared granules of examples (1,4 and 5) were used for making the Citalopram ER 60 mg tablets. The respective blend was directly compressed into tablets on a rotary tablet press (Cadmach, India) as per the target weight into suitable shape. The uncoated tablets were tested for both assay and dissolution as per the specifications.

TABLE 5
Citalopram 40 mg ER tablets

| | Example #9 | Example #10 | Example #11 |
|---|---|---|---|
| Composition | (mg/tab) | (mg/tab) | (mg/tab) |
| Citalopram hydrobromide | 50 | 50 | 50 |
| Lactose, anhydrous | 68.75 | 57.5 | — |
| HPC | 50 | — | 65 |
| Polyox ® | 26.25 | — | — |
| Kollidon ® SR | — | 87.5 | — |
| ECT10 | — | — | 80 |
| Magnesium stearate | 2.5 | 2.5 | 2.5 |
| Cab-o-sil | 2.5 | 2.5 | 2.5 |
| Total | 200 | 200 | 200 |

Procedure: The previously prepared granules of examples (1,4 and 5) were used for making the Citalopram ER 40 mg tablets. The respective blend was directly compressed into tablets on a rotary tablet press (Cadmach, India) as per the target weight into suitable shape. The uncoated tablets were tested for both assay and dissolution as per the specifications.

TABLE 6
Citalopram 20 mg ER tablets

| | Example #12 | Example #13 | Example #14 |
|---|---|---|---|
| Composition | (mg/tab) | (mg/tab) | (mg/tab) |
| Citalopram | 25 | 25 | 25 |
| hydrobromide | | | |
| Lactose, anhydrous | 34.375 | 28.75 | — |
| HPC | 25 | — | 32.5 |
| Polyox ® | 13.125 | — | — |
| Kollidon ® SR | — | 43.75 | — |
| ECT10 | — | — | 40 |
| Magnesium stearate | 1.25 | 1.25 | 1.25 |
| Cab-o-sil | 1.25 | 1.25 | 1.25 |
| Total | 100 | 100 | 100 |

Procedure: The previously prepared granules of examples (1,4 and 5) were used for making the Citalopram ER 20 mg tablets. The respective blend was directly compressed into tablets on a rotary tablet press (Cadmach, India) as per the target weight into suitable shape. The uncoated tablets were tested for both assay and dissolution as per the specifications.

### Example 15

The direct compression tablets of Escitalopram oxalate (5-50 mg), Paroxetine hydrochloride (10-50 mg), Venlafaxine hydrochloride (10-200 mg), Sertraline hydrochloride (10-200 mg), Fluoxetine hydrochloride (10-100 mg) and duloxetine hydrochloride (10-100 mg) are prepared according to the examples (1-14) described by maintaining the same percentage proportions of each ingredients other than the active ingredient.

TABLE 7
Paroxetine (10-50 mg) and Sertraline (10-200 mg) ER tablets

| | Example #16 | Example #17 | Example #18 | Example #19 |
|---|---|---|---|---|
| Composition | (mg/tab) | (mg/tab) | (mg/tab) | (mg/tab) |
| Paroxetine or | 15% | 20% | 15% | 20% |
| Sertraline HCl | | | | |
| Starch | 15% | 15% | 15% | 10 |
| HPC | 35% | — | — | — |
| Polyox ® | — | 25% | 28% | — |
| Kollidon ® SR | — | — | 40% | 35% |
| Ethylcellulose | 33% | 33% | — | — |
| Kollidon | — | — | — | 15% |
| VA 64 | | | | |
| Polyvinyl | — | — | — | 13% |
| alcohol | | | | |
| Stabilizer | — | 5% | — | 5% |
| Magnesium | 1% | 1% | 1% | 1% |
| Cab-o-sil | 1% | 1% | 1% | 1% |
| Total (%) | 100 | 100 | 100 | 100 |

Procedure: Mix the active ingredient (Paroxetine or Sertraline hydrochloride), starch with optional polyvinyl alcohol and stabilizer (ascorbic acid, hydroxypropyl betacyclodextrin, amberlite IRP 69, butylated hydroxy anisole, butylated hydroxy toluene), and granulate the mixture with acidified ethanolic solution (0.1N HCl, ethanolic) and dry them at 40° C. in a tray dryer. Blend the dry granules with release controlling polymer, glidants and lubricants. Compress the granules into tablets or fill into capsules. Optionally, prepare acidified placebo granules and dry them at 40° C. in a tray dryer. Blend the dry granules with active drug, release controlling polymer, glidants and lubricants.

TABLE 8
Duloxetine (10-100 mg) and Fluoxetine (10-100 mg) ER tablets

| | Example #20 | Example #21 | Example #22 | Example #23 |
|---|---|---|---|---|
| Composition | (mg/tab) | (mg/tab) | (mg/tab) | (mg/tab) |
| Duloxetine or | 15% | 20% | 15% | 20% |
| Fluoxetine HCl | | | | |
| Lactose | 15% | 10% | 10% | 15% |
| HPC | 10% | 15% | 20% | 25% |
| Cellulose | 10% | — | 10% | — |
| acetate | | | | |
| Ethylcellulose | 28% | 13% | 33% | 28% |
| MCC | — | 20% | — | — |
| Carbopol ® | 10% | 5% | — | — |
| 71G | | | | |
| GMS | — | 5% | — | — |
| Magnesium | 1% | 1% | 1% | 1% |
| stearate | | | | |
| Cab-o-sil | 1% | 1% | 1% | 1% |
| Eudragit | 10% | 10% | 10% | 10% |
| L30D-55 | | | | |
| Total (%) | 100 | 100 | 100 | 100 |

Procedure: Mix the active ingredient (fluoxetine or duloxetine hydrochloride) with lactose, ethylcellulose and granulate the mixture with ethanol, optionally include cellulose acetate also into the mixture before granulation (example 20 and 22). Dry the granules in a try dryer at 40° C. Blend the dry granules with release controlling polymers, glidants and lubricants. Compress the granules into tablets or fill into capsules. Optionally, mix the active ingredient with lactose, MCC, glyceryl monostearate (example 21) and granulate the mix with water. Pass the granules through extruder or spheronizer. Dry the pellets or spheroids in a try dryer at 40° C. Blend the dry granules with release controlling polymers, glidants and lubricants. Compress the granules into tablets or fill into capsules. Coat tablets or pellets with aqueous solution of eudragit in a fluid bed coater or automatic coating system at inlet temperature of 30° C. and cure the coated tablets at least for 2.0 hrs. Fill coated pellets into capsule.

TABLE 9
Venlafaxine (10-200 mg) ER tablets

| | Example #24 | Example #25 | Example #26 | Example #27 |
|---|---|---|---|---|
| Composition | (mg/tab) | (mg/tab) | (mg/tab) | (mg/tab) |
| Venlafaxine | 15% | 20% | 15% | 20% |
| hydrochlride | | | | |
| Ethylcellulose | 30% | 20% | 30% | 35% |
| Wax material | 10% | 5% | 10% | 5% |
| Cellulose | 15% | 15% | 10% | 10% |
| acetate | | | | |
| Kollidon ® SR | — | 30% | — | 15% |
| Polyox ® | — | 8% | — | 13% |
| HPC | 28% | — | 33% | — |
| Magnesium | 1% | 1% | 1% | 1% |
| stearate | | | | |
| Cab-o-sil | 1% | 1% | 1% | 1% |
| Total (%) | 100 | 100 | 100 | 100 |

Procedure: Mix the active drug with ethylcellulose, cellulose acetate and optionally with wax material (Stearic acid, cetostearyl alcohol, carnauba wax, precirol® ATO 5 or microcrystalline wax) to include in granulation. Granulate the mix with ethanol and dry the granules in a tray dryer at 30-40° C. Blend the dry granules with release controlling polymers, glidants and lubricants. Compress the granules into tablets or fill into capsules.

### Example 28

To prepare citalopram hydrobromide and escitalopram oxalate formulations by wet granulation method, follow the method described in example (16-27) by maintaining the same percentage proportions of each ingredients other than the active ingredient.

Film coating. The tablet dosage forms described in examples 1,4-14 were film coated using opadry® coating dispersion system.

### Example—29

### Film Coating of Citalopram HBr Tablets

| | 20 mg | 40 mg | 60 mg | 80 mg |
|---|---|---|---|---|
| | Qty | Qty | Qty | Qty |
| Composition | (mg/tab) | (mg/tab) | (mg/tab) | (mg/tab) |
| Citalopram ER core | 100 | 200 | 300 | 400 |
| tablets | | | | |
| Opadry ® | 3 | 6 | 9 | 12 |
| Total | 103 | 206 | 309 | 412 |

Procedure: The opadry coating system was dispersed in water and soaked for 30 min and solution was homogenized with help of homogenizer for 10 min and filtered through nylon cloth. The core tablets of were coated with above filtered solution in an automatic coating system (GAC 250, Gansons, India) with inlet temperature 60° C. and outlet temperature 40° C. until the required weight gain was achieved. The film coated tablets were dried for 20 min stored in a tight container. The coated tablets were tested for dissolution.

### Example—30

To film coat the extended release formulations of escitalopram, paroxetine, sertraline, venlafaxine, duloxetine and fluoxetine follow same steps as described in example—29. Alternatively, Replace the coating material with Opadry® II, Opadry® AMB, Kollicoat® IR and Eudragit® release controlling polymers. Prepare the solutions as per the recommendations of manufacturer in water or non-aqueous solvent system or its mixtures.

Enteric coating. The tablet dosage forms described in examples 1,4-14 were initially film coated using opadry® coating dispersion system to enhance the adsorption of enteric film onto the dosage form. The film coated tablets were further enteric coated using sureteric® coating dispersion system.

### Example—31

### Enteric Coating of Citalopram HBr Tablets

| | 20 mg | 40 mg | 60 mg | 80 mg |
|---|---|---|---|---|
| | Qty | Qty | Qty | Qty |
| Composition | (mg/tab) | (mg/tab) | (mg/tab) | (mg/tab) |
| Citalopram ER core | 100 | 200 | 300 | 400 |
| tablets | | | | |
| Opadry ® | 3 | 6 | 9 | 8 |
| Sureteric ® | 7 | 14 | 21 | 28 |
| Total | 109 | 218 | 327 | 436 |

Procedure: Both the opadry® and sureteric® coating systems were dispersed separately in water and soaked each for 30 and 60 min respectively and solutions were homogenized with help of homogenizer for 10 min. The core tablets of were precoated with opadry solution in a automatic coating system (GAC250, Gansons, India) with inlet temperature 60° C. and outlet temperature 40° C. until the required weight gain was achieved. The film coated tablets were dried for 20 min followed by coating them with filtered enteric polymer solution. The coating was continued under same pre-coating conditions until the required weight gain was achieved. The tablets were dried for 30 min and stored in a tight container. The coated tablets were tested for dissolution.

### Example—32

To enteric coat the extended release formulations of escitalopram, paroxetine, sertraline, venlafaxine, duloxetine and fluoxetine follow same steps as described in example—31. Alternatively, Replace the enteric coating material with Opadry® enteric, Acryl-Eze®, Eudragit® (E, L and S series and their mixtures) Prepare the solutions as per the recommendations of manufacturer in water or non-aqueous solvent system or its mixtures.

Dissolution studies. Dissolution studies were performed at 37° C. using USP apparatus II (Vankel Model: 600 or Hansons Research Model: SR-8 Plus).

The prepared tablet dosage forms are tested for dissolution in a USP Type II apparatus with a paddle speed of 75 rpm in various buffer media to check the pH dependent solubility/release of the drug from the dosage form and the percentage release data is shown in Table(s) 7-13.

Citalopram 80 mg ER uncoated tablets. The dissolution study of citalopram 80 mg ER uncoated dosage forms are tested using USP type II apparatus with a paddle speed of 75 rpm in a 900 ml medium of 0.1N HCl. The samples are withdrawn at various intervals and are estimated by UV at 250 nm. The percentage release data is shown in Table 10.

TABLE 10
Citalopram 80 mg tablets (uncoated, 0.1N HCl, based on example 29)

| | Example | Example | | | |
|---|---|---|---|---|---|
| | #1 | #2 | | | |
| Time | (% | (% | Example #3 | Example #4 | Example #5 |
| (hr) | Release) | Release) | (% Release) | (% Release) | (% Release) |
| 1 | 14.5 | 18.3 | 19.3 | 32.8 | 29.5 |
| 2 | 28.1 | 35.2 | 32.1 | 46.5 | 43.8 |
| 4 | 58.8 | 61.5 | 57.5 | 63.6 | 61.3 |
| 6 | 82.9 | 80.6 | 77.4 | 75.9 | 73.3 |
| 8 | 97.1 | 93.7 | 88.9 | 85.5 | 82.8 |

Citalopram 80 mg ER film coated tablets The dissolution study of citalopram 80 mg ER film coated dosage forms are tested using USP type II apparatus with a paddle speed of 75 rpm in a 900 ml medium of 0.1N HCl. The samples are withdrawn at various intervals and are estimated by UV at 250 nm. The percentage release data is shown in Table 11.

TABLE 11
Citalopram 80 mg film coated tablets (0.1N HCl, based on example 29)

| | Example #1 | Example #4 | Example #5 |
|---|---|---|---|
| Time (hr) | (% Release) | (% Release) | (% Release) |
| 1 | 16.1 | 29.1 | 35.1 |
| 2 | 28.4 | 43.0 | 54.1 |
| 4 | 57.1 | 64.5 | 80.7 |
| 6 | 80.8 | 78.4 | 98.7 |
| 8 | 93.9 | 87.7 | 104.5 |

Citalopram 80 mg ER enteric coated tablets The dissolution study of citalopram 80 mg ER enteric coated dosage forms are tested using USP type II apparatus with a paddle speed of 75 rpm in 750 ml of 0.1N HCl for 2 hrs followed by addition of 250 ml of phosphate buffer (0.02M, pH adjusted to 6.8) for remaining period of up to 8 hrs. The samples are withdrawn at various intervals and are estimated by UV at 250 nm. The percentage release data is shown in Table 12.

TABLE 12
Citalopram 80 mg Enteric coated tablets (based on example 31)

| | Example #1 | Example #4 | Example #5 |
|---|---|---|---|
| Time (hr) | (% Release) | (% Release) | (% Release) |
| 1 | 0.0 | 0.0 | 0.0 |
| 2 | 0.1 | 0.0 | 0.0 |
| 4 | 47.0 | 49.5 | 63.8 |
| 6 | 69.6 | 70.6 | 88.9 |
| 8 | 86.5 | 84.5 | 105.4 |

Citalopram 60 mg ER uncoated tablets. The dissolution study of citalopram 60 mg ER uncoated dosage forms are tested using USP type II apparatus with a paddle speed of 75 rpm in a 900 ml medium of 0.1N HCl. The samples are withdrawn at various intervals and are estimated by UV at 250 nm. The percentage release data is shown in Table 13

TABLE 13
Citalopram 60 mg tablets (uncoated, 0.1N HCl, based on example 29)

| | Example #6 | Example #7 | Example #8 |
|---|---|---|---|
| Time (hr) | (% Release) | (% Release) | (% Release) |
| 1 | 18.4 | 36.0 | 34.4 |
| 2 | 33.9 | 51.6 | 53.8 |
| 4 | 63.8 | 73.4 | 80.4 |
| 6 | 91.5 | 88.7 | 98.3 |
| 8 | 107.5 | 99.2 | 109.8 |

Citalopram 40 mg ER uncoated tablets. The dissolution study of citalopram 40 mg ER uncoated dosage forms are tested using USP type II apparatus with a paddle speed of 75 rpm in a 900 ml medium of purified water. The samples are withdrawn at various intervals and are estimated by UV at 250 nm. The percentage release data is shown in Table 14.

TABLE 14
Citalopram 40 mg tablets (uncoated, Water, based on example 29)

| | Example #9 | Example #10 | Example #11 |
|---|---|---|---|
| Time (hr) | (% Release) | (% Release) | (% Release) |
| 1 | 21.8 | 35.9 | 38.3 |
| 2 | 36.4 | 50.2 | 57.3 |
| 4 | 58.8 | 69.2 | 84.7 |
| 6 | 77.0 | 81.6 | 103.2 |
| 8 | 99.4 | 91.1 | 109.2 |

Citalopram 20 mg ER uncoated tablets. The dissolution study of citalopram 20 mg ER uncoated dosage forms are tested using USP type II apparatus with a paddle speed of 75 rpm in a 900 ml medium of 0.2M phosphate buffer (pH 6.8). The samples are withdrawn at various intervals and are estimated by UV at 250 nm. The percentage release data is shown in Table 15.

TABLE 15
Citalopram 20 mg tablets (uncoated, pH 6.8, based on example 29)

| | Example #12 | Example #13 | Example #14 |
|---|---|---|---|
| Time (hr) | (% Release) | (% Release) | (% Release) |
| 1 | 14.9 | 25.0 | 14.2 |
| 2 | 31.2 | 39.1 | 22.2 |
| 4 | 55.6 | 58.0 | 39.7 |
| 6 | 77.0 | 71.3 | 58.5 |
| 8 | 95.2 | 79.7 | 74.4 |

The release profile of coated tablets of escitalopram, paroxetine, sertraline, venlafaxine, duloxetine and fluoxetine to follow similar criteria as mentioned in table 10-15 and to analyze samples at appropriate wavelength for their measurement and the percentage of release tested under similar conditions will be as mentioned in Table 16.

TABLE 16
Percentage release requirements for extended release preparations

| | Percentage release (%) | | |
|---|---|---|---|
| Time (hr) | Uncoated | Film coated | Enteric coated |
| 1 | 10-40 | 10-40 | <10% |
| 2 | 20-60 | 20-60 | <10% |
| 4 | 40-85 | 40-85 | 30-70 |
| 8 | >70 | >70 | >70 |

Stability studies. The stability studies are conducted for illustrated examples (1-14) of citalopram hydrobromide dosage form either coated or uncoated tablets were tightly packed in a suitable container and were stored at 40° C./75% RH, 30° C./60% RH and 25° C./60% RH. The samples were withdrawn at various time points and analyzed for both dissolution and assay by HPLC method. The dissolution samples were analyzed as per the procedure mentioned under dissolution studies. The uncoated or film coated stability samples were finely grinded into powder form and extracted with 50:50 methanol: water mixture heated to 60° C. by sonication for 60 min. The samples were allowed to cool down and were filtered through 0.45 μm nylon syringe filter. The enteric coated tablets were finely grinded into powder form extracted with 25 ml of pH 6.8 buffer, sonicate for 5 min to dissolve to the enteric coat followed by extracting the samples with 50:50 methanol: water mixture heated to 60° C. by sonication for 60 min. The filtered samples were injected into HPLC. The specifications were column: Waters Xterra C₈, 150×4.6 mm, 3.5 μm; Mobile phase: methanol (20 parts): acetonitrile (20 parts): buffer (0.01M ammonium bicarbonate, pH 11, 60 parts), Wavelength: 240 nm and Flow rate: 1.0 ml/min. The sample should maintain at least 90% of potency of undegraded citalopram hydrobromide after storage for three months at about 40° C. and 75% RH. The formulations stability of escitalopram, paroxetine, sertraline, venlafaxine, duloxetine and fluoxetine are to be tested under similar conditions as described above using appropriate test conditions and assay method.

### FURTHER EXAMPLES

The invention is further illustrated according to the following further examples:

### Further Example 1

A pharmaceutical controlled release solid dosage form consisting of any newer antidepressants belongs to the category of selective serotonin reuptake inhibitors and selective serotonin norepinephrine reuptake inhibitors such as citalopram or escitalopram and any acceptable pharmaceutical salts of them for extending or controlling the release of drug in a mixture of suitable excipients used for its therapeutic effect and further to minimize dose associated side effects and such dosage form retains at least about 80% potency after storage for three months at about 40° C. and 75% RH.

### Further Example 2

The dosage form mentioned in further example 1, wherein the category of selective serotonin reuptake inhibitors belong to drugs such as citalopram, escitalopram, paroxetine, sertraline, fluoxetine and their pharmaceutically acceptable salt forms.

### Further Example 3

The dosage form mentioned in further example 1, wherein the category of selective serotonin norepinephime reuptake inhibitors can belong to drugs such as duloxetine, venlafaxine and their pharmaceutically acceptable salt forms.

### Further Example 4

The solid dosage form mentioned in further example 1 as either coated or uncoated solid tablet or capsule dosage form.

### Further Example 5

The solid dosage form mentioned in further example 4, wherein the drug is known to posses any of the following problems such as lack of stability, color formation, poor compressibility, posses either very high solubility or poor solubility, bitterness, known to interact with enteric polymers and the developed dosage form is known to address such problems other than minimizing drug associated side effects.

### Further Example 6

The dosage form mentioned in further example 4, wherein the active material is co-mixed with suitable controlled release agents and other suitable excipients to either directly compress or to granulate with suitable agents followed by a convenient coat to cover the dosage form prior to compression or filling, or to coat after making the dosage form or optionally to coat both prior and after making of the dosage form to achieve the desired release.

### Further Example 7

The dosage form mentioned in further example 4, wherein the active material is co-mixed with suitable excipients are of hydrophilic or hydrophobic nature such as diluents, binders, pH dependent or independent release controlling polymers blended with optional pore former, stabilizers or solubilizers, such mixture is blended with glidants and lubricants to compress directly to form a matrix or to fill them in the form of granules/pellets into a suitable capsule size, which is optionally film/enteric coated to cover the dosage form to achieve the desired release.

### Further Example 8

The dosage form mentioned in further example 4, wherein the active drug is mixed with pretreated placebo granules, release modifying agents, solubilizers, stabilizers, glidants and lubricants, which are blended together and compressed directly or filled into capsules. The pretreated placebo granules are prepared with suitable diluents and binders with optional acidifying agents or stabilizers included either in the blend or added to the granulating fluid to dissolve or disperse them in solution of such as water or in volatile non-aqueous solvents such as ethanol, methanol, isopropyl alcohol or in mixtures of above non-aqueous solvents with water to make placebo granules of sufficient strength and dried.

### Further Example 9

The dosage form mentioned in further example 4 wherein the active drug is physically mixed with suitable excipients are of hydrophilic or hydrophobic nature such as diluents, binders, pH dependent or independent release controlling polymers, stabilizers, solubilizers and lubricants, such mixture is
a. granulated with aqueous, non-aqueous solvents or hydroalcoholic mixtures, wherein the granulating fluid such as water, ethanol, methanol, isopropyl alcohol or mixture of such solutions can be used.
b. optionally, the granulating fluid can contain acidifying agents or stabilizers are of organic or inorganic nature suitably dissolved or dispersed in them to be effective or to ensure homogeneity during mixing.
c. optionally, the granulated mass can be extruded and spheronized without applying any external heat.
d. optionally, the drug is dissolved in above solvent system having suitable excipients such as binders, diluents, release controlling polymers and lubricants, and such solution with or with out acidifying agents or stabilizers, is spray dried directly in a fluidized system or sprayed onto placebo granules to improve their handling or release characteristics or to facilitate further optional film/enteric coating onto such dried granules.
e. optionally, the core granules, pellets or spheroids having active drug are coated with solutions having suitable excipients such as binders, diluents, release controlling polymers and lubricants, and such solution with or with out acidifying agents or stabilizers, is sprayed directly onto active granules, pellets or spheroids to improve their release characteristics or to facilitate further optional film/enteric coating onto such dried granules.
f. dried granules are blended with lubricants, glidants and optional release controlling agents, which is compressed directly or filled into capsules and such dosage form is optionally film/enteric coated further to cover the dosage form to achieve the desired release.

### Further Example 10

The solid dosage form mentioned in further example 4, which is conveniently either film or enteric coated to act as barrier for release or to protect against any additional color development/degradation products formed due to environmental factors or to mask the taste or to retard the release in acid medium, wherein the coating materials are dispersed in solutions of either aqueous/nonaqueous solvents or hydro-alcoholic mixtures thereof.

### Further Example 11

The controlled release dosage form mentioned in further example 1 consisting of either citalopram hydrobromide or escitalopram oxalate or its pharmaceutically acceptable salt thereof, with controlled release agent, suitable diluent/binder, glidants, lubricants and optional pore forming agents or stabilizers or solubilizers was developed to treat psychotic disorders or to minimize dose associated side effects during their administration.

### Further Example 12

The solid dosage form mentioned in further example 11, wherein the alternative active ingredient is paroxetine, sertraline, venlafaxine, duloxetine and fluoxetine or any of its pharmaceutically acceptable salt form with controlled release agent, suitable diluent/binder, glidants, lubricants and optional pore forming agents or stabilizers or solubilizers was developed to treat psychotic disorders or to minimize dose associated side effects during their administration.

### Further Example 13

The formulation mentioned in further example 11, the controlled release can be achieved using individual or combination of excipients known in the art to perform as barrier-forming polymer(s), erodable or insoluble material(s) of hydroxy propyl cellulose, hydroxypropyl ethyl cellulose, hydroxyethyl cellulose, hydroxyethyl methy cellulose, methylcellulose, sodium carboxymethylcellulose and calcium carboxymethylcellulose, ethylcellulose, hypermellose, polyethylene oxides of molecular weight 100,000-7,000,000 Daltons (Polyox®), polyvinyl alcohols (MW: 20,000-200,000 Daltons), substituted copolymers of polyvinyl acetate and polyvinylpyrrolidone like kollidon® VA64 and kollidon® SR, sodium alginate, carrageenan, carbomer such as carbopol 71G, 971P, 934P, and 974P, xanthan gum individually or in combination of ceratonia, locust bean gum or veegum, guar gum, gellan gum, and chitosan, dextrates, dextrins, eudragit® (RL, RS and NE grade and pH dependent release polymers such as E, L and S grade and their mixtures), eudragit® RSPO, eudragit®RLPO, cellulose acetate, cellulose acetate trimellitate, cellulose acetate butyrate, cellulose acetate propionate, cetostearyl alcohol, cetyl alcohol, glyceryl behenate derivatives like compritol® 888 ATO, precirol® ATO 5, gelucire® 44/14, gelucire® 50/13, glyceryl mono oleate, glyceryl mono stearates, glyceryl palmito stearates, lecithin, medium chain triglycerides, stearic acid, stearyl alcohol, hydrogenated vegatable oil, carnauba wax, microcrystalline wax and beeswax.

### Further Example 14

The formulation mentioned in further example 11, wherein the preferable extended release agents but not limited to hydroxy propyl cellulose, hydroxypropyl ethyl cellulose, hydroxyethyl cellulose, hydroxyethyl methy cellulose, methylcellulose, sodium carboxymethylcellulose and calcium carboxymethylcellulose, ethylcellulose, hypermellose, polyethylene oxides of molecular weight 100,000-7,000,000 Daltons (Polyox®), polyvinyl alcohols (MW: 20,000-200,000 Daltons), substituted copolymers of polyvinyl acetate and polyvinylpyrrolidone like kollidon®VA64 and kollidon® SR, sodium alginate, carbomer such as carbopol 71 G and 971 P, xanthan gum, locust bean gum, eudragit® ((RL, RS and NE grade and pH dependent release polymers such as E, L and S grade and their mixtures), eudragit® RSPO, eudragit® RLPO, cellulose acetate, cellulose acetate trimellitate, cellulose acetate butyrate, cellulose acetate propionate, cetostearyl alcohol, cetyl alcohol, glyceryl behenate derivatives like compritol® 888 ATO, precirol® ATO 5, gelucire® 44/14, gelucire® 50/13, glyceryl mono oleate, glyceryl mono stearates, stearic acid, stearyl alcohol, hydrogenated vegatable oil, carnauba wax, microcrystalline wax and beeswax.

### Further Example 15

The formulation mentioned in further example 11, consists of diluents and such materials is expected to alone or in a combination to act as binder, pore-forming agent, taste masking agent, stabilizer, solubilizer or release modifying agent, which include acacia, alginic acid, acetyltributyl citrate, acetyltriethyl citrate, beeswax, chitosan, carbomer, carageenan, carnauba wax, cellulose acetate, cetostearyl alcohol, cetyl alcohol, cellulose acetate phthalate, colloidal silicon dioxide, cyclodextrins, dibasic calcium phosphate anhydrous, carboxymethylcellulose sodium, carboxymethylcellulose calcium, crospovidone, calcium carbonate, dextran, dextrins, dextrose, ethylcellulose, fructose, guar gum, gelatin, glyceryl behenate derivatives like compritol® 888 ATO, precirol® ATO 5, gelucire® 44/14, gelucire® 50/13, glyceryl mono oleate, glyceryl mono stearates, hydroxypropyl cellulose, hydroxypropyl ethylcellulose, hydroxyethyl cellulose, hydroxyethyl methylcellulose, hypromellose, hydrogenated vegatable oil, kaolin, lactose, mannitol, maltitol, maltose, medium chain triglycerides, maltodextrin, microcrystalline cellulose, methylcellulose, magnesium stearate, magnesium aluminum silicate, microcrystalline wax, povidone, polyethylene oxide, powdered cellulose, substituted copolymers of polyvinylacetate—polyvinyl pyrrolidone, poloxamer, polydextrose, polacrilin resin, polyethylene glycol, ployoxyethylene castor oil derivatives, polyvinyl alcohol, polymethacrylates, propylene glycol alginate, pregelatinized starch, starch, stearic acid, stearyl alcohol, sucrose, saccharin, silicified microcrystalline cellulose, sorbitol, sugar spheres, sodium stearyl fumarate, sodium alginate, tragacanth, trehalose, talc, xanthan gum and xylitol.

### Further Example 16

The formulation mentioned in further example 11, wherein the binder is preferably belong to any of the following but not limited to such as acacia, alginic acid, beeswax, chitosan, carbomer such as carbopol 71G and 971P, carageenan, carnauba wax, dibasic calcium phosphate anhydrous, carboxymethylcellulose sodium, carboxymethylcellulose calcium, crospovidone, calcium carbonate, ethylcellulose, guar gum, gelatin, glyceryl behenate derivatives like compritol® 888 ATO, precirol® ATO 5, gelucire® 44/14, gelucire® 50/13, glyceryl mono oleate, glyceryl mono stearates, hydroxypropyl cellulose, hydroxypropyl ethylcellulose, hydroxyethyl cellulose, hydroxyethyl methylcellulose, hypromellose, hydrogenated vegatable oil, lactose, mannitol, microcrystalline cellulose, methylcellulose, magnesium aluminum silicate, microcrystalline wax, povidone, polyethylene oxide, powdered cellulose, substituted copolymers of polyvinylacetate—polyvinyl pyrrolidone such as kollidon® VA 64 and kollidon® SR, polyethylene glycol, poloxamer, polyvinyl alcohol, polymethacrylates, propylene glycol alginate, pregelatinized starch, starch, stearic acid, stearyl alcohol, silicified microcrystalline cellulose, sorbitol, sodium alginate, tragacanth and xanthan gum.

### Further Example 17

The formulation mentioned in further example 11, wherein preferably the pore-forming agent, taste masking agent or solubilizer belongs to any of the following but not limited to such as acacia, beeswax, chitosan, carnauba wax, cyclodextrins, cellulose acetate, carboxymethylcellulose sodium, carboxymethylcellulose calcium, crospovidone, dextran, dextrins, dextrose, fructose, guar gum, hydroxypropyl cellulose, hydroxypropyl ethylcellulose, hydroxyethyl cellulose, hydroxyethyl methylcellulose, hypromellose, lactose, mannitol, maltitol, maltose, maltodextrin, microcrystalline cellulose, methylcellulose, povidone, polyethylene oxide, powdered cellulose, poloxamer, polydextrose, polyethylene glycol, ployoxyethylene castor oil derivatives, starch, sucrose, saccharin, sorbitol, sugar spheres and xylitol.

### Further Example 18

The formulation mentioned in further example 11, wherein preferably the stabilizing agent can be an acidifying agent, complexing agent, chelating agent or an antioxidant but not limited to following such as acetic acid, adipic acid, aspartic acid, citric acid, edetic acid, fumaric acid, hydrochloric acid, lactic acid, nitric acid, malic acid, maleic acid, oxalic acid phosphoric acid, phthalic acid, sulfuric acid, succinic acid, tartaric acid, toluene sulfonic acid, and other stabilizers such as alpha tocopherol, ascorbic acid, ascorbyl palmiate, benzoic acid, butylated hydroxy anisole (BHA), butylated hydroxy toluene (BHT), cyclodextrins, disodium edetate, ion-exchange resins, isoascorbic acid, propionic acid, propyl gallate, monothioglycerol and sodium metabisulfite.

### Further Example 19

The glidant and lubricant of further example 11 are colloidal silicon dioxide, talc, magnesium stearate, stearic acid and sodium stearyl fumarate.

### Further Example 20

The formulation mentioned in further example 11, wherein the active drug comprises up 5 to 80% of dosage form and preferably in the amount of 5 mg to 300 mg of active drug.

### Further Example 21

The formulation composition of the further example 11, wherein the controlled release agent may vary from 5% to 90% w/w an individual or as 5% to 80% of each individual agent as a part of combination. The percentage weight may vary from 5% to 60% w/w of the dosage form.

### Further Example 22

The formulation composition of the further example 11, wherein the diluent/binder may vary from 5% to 60% w/w of the dosage form.

### Further Example 23

The formulation composition of the further example 11, wherein the glidant may vary from 0.5% to 3% w/w and the percentage weight of lubricant was 1-5% of the tablet weight.

### Further Example 24

The formulation composition of further example 1, wherein the core or pellet dosage form can suitably be film coated with readily dispersible coating systems or any conventional system known in the art either for identification, taste masking, aesthetic purpose or to achieve pH dependent or independent release and for stability against environmental factors and such materials as acetyl tributyl citrate, acetyl triethyl citrate, cellulose acetate, hydroxypropyl cellulose, hypromellose, a methacrylate copolymer such as eudragit® E100 or eudragit®EPO, eudragit® 30D, eudragit® (E, L and S series and their mixtures), kollicoat® IR or kollicoat® protect, kollicoat® SR 30D or EMM 30D, MAE 30 DP or MAE 100P, opadry® II, opadry® AMB and ethylcellulose (Surelease®).

### Further Example 25

The formulation composition of further example 1, wherein suitable pH dependent polymers used to retard the release from dosage form include cellulose acetate phthalate, cellulose acetate trimelliate, hypromellose phthalate, hypromellose acetate succinate, polyvinyl acetate phthalate based dispersions like Opadry® enteric, Sureteric® and Acryl-Eze®, pH dependent release polymers like eudragit® (E, L and S series or their mixtures) either directly or after mixing with suitable plasticizers, glidants, opacifying agents or coloring agents.

### Further Example 26

The formulation composition of further example 1, wherein the active drug such as citalopram hydrobromide or escitalopram oxalate stored at 40° C. and 75% RH for four to twelve weeks contains at least about 80% potency of undegraded active drug moiety.

### Further Example 27

The composition of the further example 1, wherein the active drug such as paroxetine, sertraline, venlafaxine, duloxetine and fluoxetine stored at 40° C. and 75% RH for four to twelve weeks contains at least about 80% potency of undegraded active drug moiety.

### Further Example 28

The composition of the further example 1, wherein the other newer antidepressant drug such as bupropion or fluvoxamine stored at 40° C. and 75% RH for four to twelve weeks contains at least about 80% potency of undegraded active drug moiety.

### Further Example 29

The film coated controlled release dosage form mentioned in further example 1, that exhibits the following dissolution profile when tested as per USP 27 in a USP type 2 apparatus, at 75 rpm in 1000 mL in 01.N HCl or water or phosphate buffer (0.2M, pH 6.8) at 37° C., after 1 hr the release should be between 10 to 40%, after 2 hours should be between 20 to 50%, after 4 hours should be between 40% to 70% and after 8 hours should be more than 70%.

### Further Example 30

The film coated or uncoated controlled release dosage form mentioned in further example 1, that exhibits the following dissolution profile when tested as per USP 27 in a USP type 2 apparatus, at 75 rpm in 1000 mL in 01.N HCl or water or phosphate buffer (0.2M, pH 6.8) at 37° C., after 1 hr the release should not be more than 40%, after 4 hours should not more than 70% and after 8 hours should not be less than 70%.

### Further Example 31

The enteric coated controlled release tablet dosage form mentioned in further example 1, that exhibits the following dissolution profile when tested in a USP type 2 apparatus, at 75 rpm in 750 mL in 0.1N HCl for 2 hrs and added 250 ml of 0.2M phosphate buffer and adjusted to pH to 6.8 after 2 hrs wherein the release should not be more than 10% in 2 hrs, after 4 hr the release should be between 30 to 70% and after 8 hours should be more than 70%.

### Further Example 32

The enteric coated controlled release tablet dosage form mentioned in further example 2 that exhibits the following dissolution profile when tested in a USP type 2 apparatus, at 75 rpm in 750 mL in 0.1N HCl for 2 hrs and added 250 ml of 0.2M phosphate buffer and adjusted to pH to 6.8 after 2 hrs wherein the release should not be more than 10% in 2 hrs, after 4 hr the release should not be more than 70% and after 8 hours should not be less than 70%.

### ADDITIONAL FURTHER EXAMPLES

The invention is well-illustrated according to the following additional further examples:

### Additional Further Example 1

A pharmaceutical controlled release solid dosage form comprising (a) an active pharmaceutical ingredient, comprising a selective serotonin reuptake inhibitor or a pharmaceutically acceptable salt of a selective serotonin reuptake inhibitor or a selective serotonin norepinephrine reuptake inhibitor or a pharmaceutically acceptable salt of a selective serotonin norepinephrine reuptake inhibitor or bupropion or bupropion hydrochloride or a pharmaceutically acceptable salt of bupropion, and (b) one or more excipients, wherein the release of the active pharmaceutical ingredient from the dosage form is controlled by the one or more excipients, and wherein the dosage form retains at least about 80% potency after storage for three months at about 40° C. and about 75% relative humidity.

### Additional Further Example 2

The dosage form of additional further example 1, wherein the selective serotonin reuptake inhibitor is citalopram, escitalopram, paroxetine, sertraline, or fluoxetine, the pharmaceutically acceptable salt of the selective serotonin reuptake inhibitor is a pharmaceutically acceptable salt of citalopram, escitalopram, paroxetine, sertraline, or fluoxetine, the selective serotonin norepinephrine reuptake inhibitor is duloxetine or venlafaxine, and the pharmaceutically acceptable salt of the selective serotonin norepinephrine reuptake inhibitor is a pharmaceutically acceptable salt of duloxetine or venlafaxine.

### Additional Further Example 3

The solid dosage form of additional further example 1, wherein the dosage form is a coated solid tablet or an uncoated solid tablet or a capsule or a pellet.

### Additional Further Example 4

The dosage form of additional further example 3, wherein the dosage form is manufactured by a method wherein the active pharmaceutical ingredient is admixed with a plurality of excipients, comprising one or more suitable controlled release agents and one or more other suitable excipients, to form a mixture suitable for direct compression or a mixture suitable for granulation, and wherein the mixture suitable for direct compression is directly compressed to form a directly compressed mixture or the mixture suitable for granulation is granulated with a suitable granulating agent to form a granulated mixture, and wherein the mixture suitable for direct compression, the directly compressed mixture, the mixture suitable for granulation, or the granulated mixture is optionally coated with a suitable coating agent to form a coated mixture suitable for direct compression, a coated directly compressed mixture, a coated mixture suitable for granulation, or a coated granulated mixture.

### Additional Further Example 5

The dosage form of additional further example 4, wherein the plurality of excipients comprise one or more diluents, one or more binders, one or more release-controlling polymers, one or more pore-forming agents, one or more stabilizers, or one or more solubilizers, and wherein (a) the mixture suitable for direct compression comprises one or more glidants and one or more lubricants, and wherein the directly compressed mixture or the coated directly compressed mixture is placed into a capsule of suitable size, and wherein the capsule is optionally film-coated or enteric-coated, or (b) the granulated mixture or the coated granulated mixture is compressed to form a tablet.

### Additional Further Example 6

The dosage form of additional further example 4, wherein the plurality of excipients are formed into placebo granules, and wherein the placebo granules are prepared by a method comprising the steps of (a) admixture of the plurality of excipients with one or more solvents and (b) removal of excess amounts of the one or more solvents by drying or evaporation.

### Additional Further Example 7

The dosage form of additional further example 4, wherein the active phamaceutical ingredient is admixed with the plurality of excipients to form an active-excipient mixture, and wherein the active-excipient mixture is granulated with a granulating fluid comprising an aqueous solvent, a non-aqueous solvent or a hydroalcoholic mixture to form a granulated mass, and wherein excess solvent is removed from the granulated mass by drying or evaporation to form one or more granules.

### Additional Further Example 8

The dosage form of clam 7, wherein the granulating fluid comprises water, ethanol, methanol, or isopropyl alcohol; wherein the granulating fluid optionally comprises an acidifying agent or a stabilizer; wherein the granulated mass is optionally extruded or spheronized in a process wherein no exogenous heat is applied to the granulated mass; wherein the active pharmaceutical ingredient is optionally dissolved in a solvent system prior to admixture with the plurality of excipients; wherein optionally the active-excipient mixture is sprayed onto at least a portion of the granulated mass; and wherein optionally the granules are blended with one or more lubricants, one or more glidants and, optionally, one or more release-controlling agents, to form a granule-excipient mixture, and wherein the granule-excipient mixture is compressed directly to form a compressed mass or placed into one or more capsules to form an encapsulated mass, and wherein the compressed mass or the encapsulated mass is optionally film-coated or enteric-coated.

### Additional Further Example 9

The dosage form of additional further example 4, wherein (a) the plurality of excipients are admixed with the active pharmaceutical ingredient and one or more solvents to form a sprayable mixture, and (b) the sprayable mixture is sprayed to form a mixture spray, from which mixture spray excess one or more solvents are permitted to evaporate, and which mixture spray, upon evaporation of the excess one or more solvents, forms one or more coated particles.

### Additional Further Example 10

The solid dosage form of additional further example 1, wherein the active pharmaceutical ingredient comprises citalopram hydrobromide, escitalopram oxalate, or a pharmaceutically acceptable salt of citalopram or escitalopram.

### Additional Further Example 11

The solid dosage form of additional further example 1, wherein the one or more excipients comprise at least one member of the group consisting of controlled release agents, diluents, binders, glidants, lubricants, pore-forming agents, stabilizers, and solubilizers.

### Additional Further Example 12

The solid dosage form of additional further example 1, wherein the dosage form consists of, by mass, from about 5% to about 80% active pharmaceutical ingredient.

### Additional Further Example 13

The solid dosage form of additional further example 1, wherein the solid dosage form exhibits a dissolution profile, when tested as per USP 27 in a USP Type 2 apparatus, at 75 rpm in 1000 mL in 0.1 N HCl or water or phosphate buffer (0.2M, pH 6.8) at 37° C., wherein after 1 hour the release of active pharmaceutical ingredient is between about 10% and about 40%, after 2 hours the release of active pharmaceutical ingredient is between about 20% and about 60%, after 4 hours the release of active pharmaceutical ingredient is between about 40% and about 85%, and after 8 hours the release of active pharmaceutical ingredient is more than 70%.

### Additional Further Example 14

The solid dosage form of additional further example 1, wherein the solid dosage form exhibits a dissolution profile, when tested as per USP 27 in a USP Type 2 apparatus, at 75 rpm in 1000 mL in 0.1 N HCl or water or phosphate buffer (0.2M, pH 6.8) at 37° C., wherein after 1 hour the release of active pharmaceutical ingredient is not more than about 40%, after 4 hours the release of active pharmaceutical ingredient is not more than about 85%, and after 8 hours the release of active pharmaceutical ingredient is not less than about 70%.

### Additional Further Example 15

The solid dosage form of additional further example 1, wherein the solid dosage form exhibits a dissolution profile, when tested in a USP Type 2 apparatus, at 75 rpm in 750 mL in 0.1N HCl for 2 hrs and added 250 ml of 0.2M phosphate buffer and adjusted to pH to 6.8 after 2 hrs at 37° C., wherein after 2 hours the release of active pharmaceutical ingredient is not more than about 10%, after 4 hours the release of active pharmaceutical ingredient is between about 30% and about 70%, and after 8 hours the release of active pharmaceutical ingredient is not less than about 70%.

### Additional Further Example 16

The solid dosage form of additional further example 1, wherein the solid dosage form exhibits a dissolution profile, when tested in a USP Type 2 apparatus, at 75 rpm in 750 mL in 0.1N HCl for 2 hrs and added 250 ml of 0.2M phosphate buffer and adjusted to pH to 6.8 after 2 hrs at 37° C., wherein after 2 hours the release of active pharmaceutical ingredient is not more than about 10%, after 4 hours the release of active pharmaceutical ingredient is not more than about 70%, and after 8 hours the release of active pharmaceutical ingredient is not less than about 70%.

It is to be understood that the invention is not limited to the exact details of operation, or to the exact compositions, methods, procedures or embodiments shown and described, as obvious modifications and equivalents will be apparent to one skilled in art, and the invention is therefore to be limited only by the full scope which can fairly, legally and equitably be accorded to the appended claims.

1. A pharmaceutical controlled release solid dosage form comprising (a) an active pharmaceutical ingredient, comprising a selective serotonin reuptake inhibitor or a pharmaceutically acceptable salt of a selective serotonin reuptake inhibitor or a selective serotonin norepinephrine reuptake inhibitor or a pharmaceutically acceptable salt of a selective serotonin norepinephrine reuptake inhibitor or bupropion or bupropion hydrochloride or a pharmaceutically acceptable salt of bupropion, and (b) one or more excipients, wherein the release of the active pharmaceutical ingredient from the dosage form is controlled by the one or more excipients, and wherein the dosage form retains at least about 80% potency after storage for three months at about 40° C. and about 75% relative humidity.

2. The dosage form of claim 1, wherein the selective serotonin reuptake inhibitor is citalopram, escitalopram, paroxetine, sertraline, or fluoxetine, the pharmaceutically acceptable salt of the selective serotonin reuptake inhibitor is a pharmaceutically acceptable salt of citalopram, escitalopram, paroxetine, sertraline, or fluoxetine, the selective serotonin norepinephrine reuptake inhibitor is duloxetine or venlafaxine, and the pharmaceutically acceptable salt of the selective serotonin norepinephrine reuptake inhibitor is a pharmaceutically acceptable salt of duloxetine or venlafaxine.

3. The solid dosage form of claim 1, wherein the dosage form is a coated solid tablet or an uncoated solid tablet or a capsule or a pellet.

4. The dosage form of claim 3, wherein the dosage form is manufactured by a method wherein the active pharmaceutical ingredient is admixed with a plurality of excipients, comprising one or more suitable controlled release agents and one or more other suitable excipients, to form a mixture suitable for direct compression or a mixture suitable for granulation, and wherein the mixture suitable for direct compression is directly compressed to form a directly compressed mixture or the mixture suitable for granulation is granulated with a suitable granulating agent to form a granulated mixture, and wherein the mixture suitable for direct compression, the directly compressed mixture, the mixture suitable for granulation, or the granulated mixture is optionally coated with a suitable coating agent to form a coated mixture suitable for direct compression, a coated directly compressed mixture, a coated mixture suitable for granulation, or a coated granulated mixture.

5. The dosage form of claim 4, wherein the plurality of excipients comprise one or more diluents, one or more binders, one or more release-controlling polymers, one or more pore-forming agents, one or more stabilizers, or one or more solubilizers, and wherein (a) the mixture suitable for direct compression comprises one or more glidants and one or more lubricants, and wherein the directly compressed mixture or the coated directly compressed mixture is placed into a capsule of suitable size, and wherein the capsule is optionally film-coated or enteric-coated, or (b) the granulated mixture or the coated granulated mixture is compressed to form a tablet.

6. The dosage form of claim 4, wherein the plurality of excipients are formed into placebo granules, and wherein the placebo granules are prepared by a method comprising the steps of (a) admixture of the plurality of excipients with one or more solvents and (b) removal of excess amounts of the one or more solvents by drying or evaporation.

7. The dosage form of claim 4, wherein the active phamaceutical ingredient is admixed with the plurality of excipients to form an active-excipient mixture, and wherein the active-excipient mixture is granulated with a granulating fluid comprising an aqueous solvent, a non-aqueous solvent or a hydroalcoholic mixture to form a granulated mass, and wherein excess solvent is removed from the granulated mass by drying or evaporation to form one or more granules.

8. The dosage form of clam 7, wherein the granulating fluid comprises water, ethanol, methanol, or isopropyl alcohol; wherein the granulating fluid optionally comprises an acidifying agent or a stabilizer; wherein the granulated mass is optionally extruded or spheronized in a process wherein no exogenous heat is applied to the granulated mass; wherein the active pharmaceutical ingredient is optionally dissolved in a solvent system prior to admixture with the plurality of excipients; wherein optionally the active-excipient mixture is sprayed onto at least a portion of the granulated mass; and wherein optionally the granules are blended with one or more lubricants, one or more glidants and, optionally, one or more release-controlling agents, to form a granule-excipient mixture, and wherein the granule-excipient mixture is compressed directly to form a compressed mass or placed into one or more capsules to form an encapsulated mass, and wherein the compressed mass or the encapsulated mass is optionally film-coated or enteric-coated.

9. The dosage form of claim 4, wherein (a) the plurality of excipients are admixed with the active pharmaceutical ingredient and one or more solvents to form a sprayable mixture, and (b) the sprayable mixture is sprayed to form a mixture spray, from which mixture spray excess one or more solvents are permitted to evaporate, and which mixture spray, upon evaporation of the excess one or more solvents, forms one or more coated particles.

10. The solid dosage form of claim 1, wherein the active pharmaceutical ingredient comprises citalopram hydrobromide, escitalopram oxalate, or a pharmaceutically acceptable salt of citalopram or escitalopram.

11. The solid dosage form of claim 1, wherein the one or more excipients comprise at least one member of the group consisting of controlled release agents, diluents, binders, glidants, lubricants, pore-forming agents, stabilizers, and solubilizers.

12. The solid dosage form of claim 1, wherein the dosage form consists of, by mass, from about 5% to about 80% active pharmaceutical ingredient.

13. The solid dosage form of claim 1, wherein the solid dosage form exhibits a dissolution profile, when tested as per USP 27 in a USP Type 2 apparatus, at 75 rpm in 1000 mL in 0.1 N HCl or water or phosphate buffer (0.2M, pH 6.8) at 37° C., wherein after 1 hour the release of active pharmaceutical ingredient is between about 10% and about 40%, after 2 hours the release of active pharmaceutical ingredient is between about 20% and about 60%, after 4 hours the release of active pharmaceutical ingredient is between about 40% and about 85%, and after 8 hours the release of active pharmaceutical ingredient is more than 70%.

14. The solid dosage form of claim 1, wherein the solid dosage form exhibits a dissolution profile, when tested as per USP 27 in a USP Type 2 apparatus, at 75 rpm in 1000 mL in 0.1 N HCl or water or phosphate buffer (0.2M, pH 6.8) at 37° C., wherein after 1 hour the release of active pharmaceutical ingredient is not more than about 40%, after 4 hours the release of active pharmaceutical ingredient is not more than about 85%, and after 8 hours the release of active pharmaceutical ingredient is not less than about 70%.

15. The solid dosage form of claim 1, wherein the solid dosage form exhibits a dissolution profile, when tested in a USP Type 2 apparatus, at 75 rpm in 750 mL in 0.1N HCl for 2 hrs and added 250 ml of 0.2M phosphate buffer and adjusted to pH to 6.8 after 2 hrs at 37° C., wherein after 2 hours the release of active pharmaceutical ingredient is not more than about 10%, after 4 hours the release of active pharmaceutical ingredient is between about 30% and about 70%, and after 8 hours the release of active pharmaceutical ingredient is not less than about 70%.

16. The solid dosage form of claim 1, wherein the solid dosage form exhibits a dissolution profile, when tested in a USP Type 2 apparatus, at 75 rpm in 750 mL in 0.1N HCl for 2 hrs and added 250 ml of 0.2M phosphate buffer and adjusted to pH to 6.8 after 2 hrs at 37° C., wherein after 2 hours the release of active pharmaceutical ingredient is not more than about 10%, after 4 hours the release of active pharmaceutical ingredient is not more than about 70%, and after 8 hours the release of active pharmaceutical ingredient is not less than about 70%.
